(19) United States
(12) Patent Application Publication
(10) Pub. No.: US 2014/0147418 A1
(43) Pub. Date: May 29, 2014

(54) AAV MEDIATED CTLA-4 GENE TRANSFER TO TREAT SJOGREN'S SYNDROME

(71) Applicants: John A. Chiorini, Dayton, MD (US); Hongen Yin, Bethesda, MD (US)

(72) Inventors: John A. Chiorini, Dayton, MD (US); Hongen Yin, Bethesda, MD (US)

(73) Assignee: THE US OF AMERICA, AS REPRESENTED BY THE SECRETARY DEPARTMENT OF HEALTH AND HUMAN SERVICES, Bethesda, MD (US)

(21) Appl. No.: 14/111,604

(22) PCT Filed: Apr. 13, 2012
(86) PCT No.: PCT/US12/33556

§ 371 Date: Jan. 13, 2014

Related U.S. Application Data
(60) Provisional application No. 61/476,168, filed on Apr. 15, 2011.

(51) Int. Cl. A61K 47/48

(52) U.S. Cl. CPC A61K 47/48369
USPC 424/93.6; 536/23.4; 435/320.1; 435/235.1

(57) ABSTRACT

The invention relates to a gene transfer-based method to protect a subject from Sjogren's syndrome. The method comprises administering to the subject an AAV virion comprising an AAV vector that encodes a soluble CTLA-4 (sCTLA-4) protein. Also provided are sCTLA-4 proteins and nucleic acid molecules that encode such sCTLA-4 proteins. Also provided are AAV vectors and AAV virions that encode a sCTLA-4 protein.

### FIELD

The present invention relates to the use of gene therapy to protect a subject from Sjögren's syndrome. More specifically, the present invention relates to adeno-associated virus vectors and virions that encode an extracellular domain of the cytotoxic T lymphocyte 4 antigen and their use to protect a subject from Sjögren's syndrome.

### BACKGROUND/INTRODUCTION

Sjögren's syndrome is a systemic autoimmune disease in which immune cells attack and destroy the exocrine glands that produce saliva and tears. Sjögren's syndrome can also affect multiple organs, including kidneys and lungs. It is estimated that approximately 4 million people in the United States suffer from Sjögren's syndrome. Nine out of ten Sjögren's patients are women, with the average age of onset being in the late 40s. Sjögren's syndrome can occur in all age groups of both women and men. Sjögren's syndrome (SS) can occur independently, referred to as primary Sjögren's syndrome (pSS), or may develop years after the onset of an associated rheumatic disorder, referred to as secondary Sjögren's syndrome. The prevalence of primary Sjögren's syndrome varies from about 0.05% to 5% of the population, and the incidence of cases diagnosed by a doctor has been reported to be about 4 per 100,000 people a year (Kok et al., 2003, Ann Rhem Dis 62, 11038-1046).

Xerostomia (dry mouth) and xerophthalmia (conjunctivitis sicca, dry eyes) are hallmarks of Sjögren's syndrome (SS) (Fox et al., 1985, Lancet 1, 1432-1435). Immunologically-activated or apoptotic glandular epithelial cells that expose autoantigens in predisposed individuals might drive autoimmune-mediated tissue injury (see, e.g., Voulgarelis et al, 2010m Nat Rev Rheumatol 6, 529-537; Xanthou et al, 1999, Clin Exp Immunol 118, 154-163). Immune activation is typically presented as focal, mononuclear (T, B and macrophage) cell infiltrates proximal to the ductal epithelial cells (epithelitis) and forms sialadenitis (see, e.g., Voulgarelis et al., ibid.). Though the pathogenetic mechanism for this autoimmune exocrinopathy has not been fully elucidated, it has been shown that CD4+ T-lymphocytes constitute 60-70 percent of the mononuclear cells infiltrating the glands (see, e.g., Skopouli et al., 1991, J Rheumatol 18, 210-214). Abnormal activation of proinflammatory Th1 (see, e.g., Bombardierei et al., 2004, Arthritis Res Ther 6, R447-R456; Vosters et al., 2009, Arthritis Rheum 60, 3633-3641) and Th17 (see, e.g., Nguyen et al., 2008, Arthritis and Rheumatism 58, 734-743) cells have been reported to be central to induction of SS in either human or animal models.

Activation of Th1 and Th17 cells is initiated by antigen presentation, which requires the engagement not only of the T-cell receptor (TCR) to MHC molecules from antigen presenting cells (APCs), but also appropriate costimulatory signaling (see, e.g., Smith-Garvin et al., 2009, Ann Rev Immunol 27, 591-619). One of the crucial pathways of costimulation is the interaction of CD28 on the T cell with B7.1 (CD80)/B7.2 (CD86) on antigen presenting cells. Cytotoxic T-lymphocyte antigen 4 (CTLA-4; also referred to as CD152) displays a wide range of activities in immune tolerance. The main function of CTLA-4 is to bind to B7 and compete for its interaction with CD28, thereby shutting down the B7:CD28 pathway and subsequently initiating the deactivation of the T cell response and maintaining immune homostasis (see, e.g., Perkins et al., 1996, J Immunol 156, 4154-4159). Moreover, CTLA-4 is constitutively expressed on CD4+CD25+Foxp3+natural regulatory T cells (nTreg), which play a crucial role in immune tolerance and ultimately protection from autoimmune disease (see, e.g., Sakaguchi et al., 2006, Immunological Reviews 212, 8-27). CTLA-4 is required by nTreg cells for suppressing the immune responses by affecting the potency of APCs to activate effective T cells (see, e.g., Wing et al., 2008, Science 322, 271-275; Takahashi et al., 2000, J Exp Med 192, 303-310). It is known that T cell autoimmunity is controlled by the balances between Th17/Treg cells (see, e.g., Eisenstein et al., 2009, Pediatric Research 65, 26R-31R) and Th1/Th2 cells (see, e.g., Nicholson et al., 1996, Current Opinion Immunol 8, 837-842). Thus CTLA-4 could represent an important therapeutic target, shifting the T cell balance from proinflammatory T17 and/or Th1 towards suppressing Treg and/or Th2 cells.

Abatacept (trade name ORENCIA®, also referred to as CTLA4-Ig) is a recombinant fusion protein of the extracellular domain of CTLA-4 and an immunoglobulin, which is licensed in the United States for the treatment of rheumatoid arthritis in the case of inadequate response to anti-tumor necrosis factor-alpha (TNF-α) therapy (Genovese et al., 2005, N Engl J Med 353, 1114-1123). Abatacept, which contains the CTLA-4 high-affinity binding site for B7, works by binding to B7 protein on APCs and preventing them from delivering the costimulatory signal to T cells, thus preventing the full activation of T cells (see, e.g., Moreland et al., 2006, Nat Rev Drug Discov 5, 185-186).

The immunosuppressive effect of CTLA4Ig is, however, not limited to T cells: Cre/loxP-mediated CTLA4Ig gene transfer has been shown to induce B cell suppression (see, e.g., Izawa et al., 2006, Cardiovasc Res 69, 289-297). Treatment of synovial macrophages from rheumatoid arthritis patients in vitro led to suppression of macrophages (see, e.g., Cutolo et al., 2009, Arthritis Res Ther 11, R176). Suppression of B cells and macrophages as well as T cells suggest an expanded inhibitory role for CTLA4-Ig on autoimmunity.

It has also been noted that epithelial cells of minor salivary glands of patients with Sjögren's syndrome express costimulatory molecules B7.1 (CD80) and B7.2 (CD86) (Matsumura et al., 2001, Ann Rheum Dis 60, 473-482). Correspondingly different haplotypes of CTLA-4 have been found to be associated with increased susceptibility to pSS and with some extra-glandular manifestations of the disease (Downie-Doyle et al., 2006, Arthritis Rheum 54, 2434-2340).

At least some treatments that have proven effective for certain autoimmune diseases, such as rheumatoid arthritis, have not proven effective for Sjögren's syndrome. For example, anti-tumor necrosis factor (TNF) agents have been shown to have beneficial effects in the treatment of rheumatoid arthritis as well as in other inflammatory arthritides and diseases. Etanercept (trade name ENBREL), a fusion protein of soluble TNF receptor 2 and the Fc region of immunoglobulin IgG1, is marketed for a number of such conditions. However, etanercept has been shown to be ineffective in a clinical trial of patients with Sjögren's syndrome (see, e.g., Moutsopoulos et al., 2008, Ann Rheum Dis 67, 1437-1443). In addition, administration of an AAV vector encoding soluble TNF receptor 1-Fc fusion protein to the salivary glands of a murine model of Sjögren's syndrome has been shown to have a negative effect on salivary gland function (see, e.g., Vosters et al., 2009, Arthritis Res Ther 11, R189).

There still remains a need for an effective composition to protect subjects from Sjögren's syndrome.

### SUMMARY

The disclosure provides a gene transfer-based method to protect a subject from Sjögren's syndrome. The method comprises administering to the subject an AAV virion comprising an AAV vector that encodes a soluble CTLA-4 (sCTLA-4) protein. Also provided are methods to produce such sCTLA-4 proteins, AAV vectors, and AAV virions. Also provided are nucleic acid molecules that encode sCTLA-4 proteins of the embodiments and uses thereof.

The disclosure provides an AAV vector that encodes a fusion protein comprising a sCTLA-4 protein and an immunoglobulin fusion segment. The disclosure also provides an AAV virion that comprises an AAV vector that encodes a fusion protein comprising a sCTLA-4 protein and an immunoglobulin fusion segment. Also provided are AAV vectors that encode other sCTLA-4 proteins of the embodiments, and AAV virions that comprise such AAV vectors.

The disclosure provides a treatment for Sjögren's syndrome. Such a treatment comprises an AAV virion comprising an AAV vector that encodes a sCTLA-4 protein. Administration of such a treatment to a subject protects the subject from Sjögren's syndrome.

The disclosure also provides a preventative for Sjögren's syndrome. Such a preventative comprises an AAV virion comprising an AAV vector that encodes a sCTLA-4 protein. Administration of such a preventative to a subject protects the subject from Sjögren's syndrome.

The disclosure provides a salivary gland cell transfected with an AAV vector that encodes a sCTLA-4 protein. The salivary gland cell can be that of a subject with Sjögren's syndrome.

The disclosure also provides an AAV virion comprising an AAV vector that encodes a sCTLA-4 protein for the treatment or prevention of Sjögren's syndrome. Also provided is the use of an AAV virion comprising an AAV vector that encodes a sCTLA-4 protein for the manufacture of a medicament to protect a subject from Sjögren's syndrome.

### BRIEF DESCRIPTION OF THE FIGURES

FIG. 1 demonstrates in vitro expression and activity of CTLA4IgG. In FIG. 1A, in vitro expression of CTLA4IgG was detected by western blotting of media from pAAV2-CTLA4IgG-transfected cells (lane 3). As a control, a purified recombinant mouse CTLA4/Fc was also run on the gel (lane 2). In FIG. 1B, the biological activity assay for CTLA4IgG to bind and block B7.1 was determined by incubating with medium from either naïve HEK-293 cells (column 1) or from cells transfected with pAAV2-CTLA4IgG (column 2). Unbound B7.1 was then tested by flow cytometry using an antibody to B7.1. Data shown are mean from 3 independent experiments (*, P=0.0400). Unpaired student-t test was used in the analysis.

FIG. 2 demonstrates in vivo expression of sCTLA-4 fusion protein CTLA4IgG in salivary glands from C57BL/6.NOD-Aec1Aec2 mice. A sandwich ELISA was developed to detect expression of CTLA4IgG in homogenates of submandibular salivary glands (FIG. 2A) and serum (FIG. 2B). Data shown were mean±SEM from each group. Mice cannulated with AAV virion of the embodiments AAV2-CTLA4IgG (n=6, pooled into 2 samples/group) had significant levels of CTLA4IgG protein compared with mice that received virion AAV2-LacZ (n=7, pooled into 2 samples/group): In the salivary glands (**, P=0.0003) and serum (*, P=0.0102). Unpaired student-t test was used in the analysis.

FIG. 3 demonstrates stimulated saliva and tear flow rates in treated C57BL16.NOD-Aec1Aec2 mice. Saliva and tears were collected as described in the Examples herein. Data shown are the mean±SEM (n=6 in AAV2-LacZ group and n=7 in AAV2-CTLA4IgG group). Unpaired student t-test was used in the analysis. FIG. 3A shows that mice treated with an AAV virion expressing CTLA4IgG showed protection from loss of gland activity. Saliva was collected as described in the Examples herein over a 10-minute period after stimulation with 0.5-mg/kg body weight pilocarpine and tear flow samples were collected over a 20-second period after injection of pilocarpine (4.5 mg/kg body weight). AAV2-LacZ mice showed decreased saliva flow rates on weeks 16, 22, 26, and 30 (*, P=0.0428, 0.0217, 0.0292, 0.0128 respectively) compared with the baseline saliva collection on week 6 (n=9, 5.933±0.2969). AAV virion AAV2-CTLA4IgG treated mice had a slight decrease of saliva flow rate that was not significant at 16 weeks (P=0.2057). Saliva flow rate of AAV2-CTLA4IgG treated mice increased to baseline level by 22 weeks (6.13±0.92 μL/g 10 mins). AAV2-CTLA4IgG treated mice had increased saliva flow rate compared with AAV2-LacZ treated mice by 30 weeks (P=0.0232). FIG. 3B shows that delivery of AAV virion AAV2-CTLA4IgG resulted in an increase in tear flow rate (mean±SEM) by 30 weeks compared with control mice (P=0.1316).

FIG. 4 demonstrates results of histological examination of salivary glands administered AAV virions of the embodiments. Salivary gland histology was examined at the end of the study (30 weeks of age). CD3+T and B220+B cell immunofluorescence staining, as well as CD11c and F4/80 immunochemistry staining for dendritic cells (DCs) and macrophages was done as described in the Examples herein. Panels show representative immuno-fluorescence staining of salivary of B and T cells in salivary glands from mice cannulated with AAV2-LacZ (n=6) or AAV2-CTLA4IgG vector (n=7) (FIG. 4A, FIG. 4B, and FIG. 4C) (Blue arrows) and enumeration (mean±SEM) (FIG. 4D) of B and T cells in salivary glands from LacZ- and CTLA4IgG-treated mice; immunohistochemical staining and enumeration (mean±SEM) of CD11c+DCs (FIG. 4E, FIG. 4F, FIG. 4G, and FIG. 4H) and F4/80+ macrophages (FIG. 4I, FIG. 4J, FIG. 4K, and FIG. 4L) (Black arrows) in salivary glands from LacZ- and CTLA4IgG-treated C57BL/6.NODAec1Aec2 mice. A statistical decrease in the enumeration of T cells was shown in the salivary glands from CTLA4 overexpressing mice compared to the LacZ-treated (P=0.0464). A trend, was shown (P=0.3024) for decrease in the enumeration of B cells. Significant down-regulation of both CD11c+ dendritic cells and F4/80+macrophages was seen in the salivary glands from CTLA4IgG-treated mice compared with control (two asterisks, P≦0.01). Unpaired student-t test was used in this analysis.

FIG. 5 demonstrates serum anti-nuclear antibody productions in C57BL/6.NOD-Aec1Aec2 mice. Serum samples were analyzed for anti-Ro (SSA) (FIG. 5A) and anti-La (SSB) (FIG. 5B) antibody expression in serum from AAV2-LacZ (n=6) and AAV2-CTLA4IgG (n=7) treated mice by ELISA as described in the Examples herein. Data shown are mean±SEM from duplicate tests of pooled samples from each group. Unpaired student's t-test was used for statistical analysis. No statistical significant difference was detected. (P=0.9586 and P=0.4158 respectively).

FIG. 6 is a schematic map of AAV vector pAAV2-CMV-mCTLA4-hIgG (SEQ ID NO:1). The R ITR spans nucleotides 5369 through 5487. The CMV promoter domain spans nucleotides 5498-5922. The nucleic acid molecule encoding the mouse soluble CTLA-4 domain joined to the human immunoglobulin fusion segment spans nucleotides 283 through 2010. The polyadenylation site spans nucleotides 2565-2678. The L ITR spans nucleotides 2673 through 2802.

### DETAILED DESCRIPTION

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the claims.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It should be understood that as used herein, the term “a” entity or “an” entity refers to one or more of that entity. For example, a nucleic acid molecule refers to one or more nucleic acid molecules. As such, the terms “a”, “an”, “one or more” and “at least one” can be used interchangeably. Similarly the terms “comprising”, “including” and “having” can be used interchangeably.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed. In addition, all sub-combinations are also specifically embraced by the present invention and are disclosed herein just as if each and every such sub-combination was individually and explicitly disclosed herein.

The disclosure provides a novel gene therapy to protect a subject from Sjögren's syndrome. The inventors discovered that administration of an adeno-associated virus (AAV) virion comprising an AAV vector that encodes an extracellular domain of a cytotoxic T-lymphocyte antigen 4 (sCTLA-4) protein to a subject protects that subject from Sjögren's syndrome. This discovery is surprising because, even though a soluble CTLA-4 protein, such as the fusion protein CTLA4Ig (abatacept), can be used to treat the autoimmune disease rheumatoid arthritis, it would not necessarily be expected that such a protein could treat Sjögren's syndrome. As described above, even though TNF inhibitors have been shown to be effective against rheumatoid arthritis, they are not effective against Sjögren's syndrome. At least some treatments that have proven effective for certain autoimmune diseases, such as rheumatoid arthritis, have not proven effective for Sjögren's syndrome. For example, anti-tumor necrosis factor (TNF) agents have been shown to have beneficial effects in the treatment of rheumatoid arthritis as well as in other inflammatory arthritides and diseases. Etanercept (trade name ENBREL), a fusion protein of soluble TNF receptor 2 and the Fc region of immunoglobulin IgG1, is marketed for a number of such conditions. However, etanercept has been shown to be ineffective in a clinical trial of patients with Sjögren's syndrome (see, e.g., Moutsopoulos et al., 2008, Ann Rheum Dis 67, 1437-1443). In addition, administration of an AAV vector encoding soluble TNF receptor 1-Fc fusion protein to the salivary glands of a murine model of Sjögren's syndrome has been shown to have a negative effect on salivary gland function (see, e.g., Vosters et al., 2009, Arthritis Res Ther 11, R189).

### Proteins

As used herein, a soluble CTLA-4 protein, also referred to as a sCTLA-4 protein, is any protein that exhibits activity of the extracellular domain of a cytotoxic T-lymphocyte antigen-4, such as the ability to bind to a B7 protein, such as a B7 protein on an antigen-presenting cell (e.g., CD80 or CD86). A sCTLA-4 protein can have a wild-type CTLA-4 sequence (i.e., it has the same amino acid sequence as the extracellular domain of a natural CTLA-4), can be a portion of the extracellular domain of a natural CTLA-4, or can be a mutant of the extracellular domain of a natural CTLA-4, provided that such a portion or mutant retains the ability to bind to a B7 protein.

In one embodiment, a sCTLA-4 protein comprises the entire extracellular domain of a natural CTLA-4. In one embodiment, a sCTLA-4 protein is a portion of the extracellular domain of a natural CTLA-4, wherein such portion retains the ability to bind to a B7 protein. In one embodiment, a sCTLA-4 protein is a mutant of the extracellular domain of a natural CTLA-4, wherein such mutant retains the ability to bind to a B7 protein. In one embodiment, a sCTLA-4 protein is a portion of a mutant of the extracellular domain of a natural CTLA-4, wherein such sCTLA-4 protein retains the ability to bind to a B7 protein.

Methods to produce portions and mutants, such as conservative mutants, are known to those skilled in the art. Assays to determine binding between a sCTLA-4 protein and a B7 protein are known to those skilled in the art; see, for example, Morton et al., 1996, J Immunol 156, 1047-1054. The structure and position of the binding site on CTLA-4 for B7-2 has also been elucidated; see, for example, Schwartz et al., 2001, Nature 410, 604-608. Thus, one skilled in the art can produce portions or mutants of a sCTLA-4 protein that bind to B7 protein without undue experimentation. Binding between a sCTLA-4 protein of the embodiments and a B7 protein on an antigen presenting cell is sufficient to down-regulate a Th1-mediated immune response.

A sCTLA-4 protein of the embodiments can be derived from any species that expresses a functional cytotoxic T-lymphocyte antigen-4. A sCTLA-4 protein can have the sequence of a human or other mammalian CTLA-4 extracellular domain or portion thereof. Examples include, but are not limited to, murine, feline, canine, equine, bovine, ovine, porcine or other companion animal, other zoo animal, or other livestock CTLA-4 extracellular domain or portion thereof. In one embodiment, a sCTLA-4 protein has the amino acid sequence of a human CTLA-4 extracellular domain or portion thereof. An example of a human-derived sCTLA-4 amino acid sequence is that depicted in SEQ ID NO:5. In one embodiment, a sCTLA-4 protein has the amino acid sequence of a murine CTLA-4 extracellular domain or portion thereof. An example of a murine-derived sCTLA-4 amino acid sequence is that depicted in SEQ ID NO:4. In one embodiment, a sCTLA-4 protein is derived from the species that is being protected from Sjögren's syndrome. In one embodiment, a sCTLA-4 protein is derived from a species for which the protein is not immunogenic in the subject being protected from Sjögren's syndrome.

One embodiment of the disclosure is a sCTLA-4 protein joined to a fusion segment; such a protein is referred to as a sCTLA-4 fusion protein. Such a protein has a sCTLA-4 protein domain (also referred to herein as a sCTLA-4 domain) and a fusion segment. A fusion segment is an amino acid segment of any size that can enhance the properties of a sCTLA-4 protein; a fusion segment of the embodiments can, for example, increase the stability of a sCTLA-4 protein, add flexibility or enable multimerization, e.g., dimerization. Examples of fusion segments include, without being limited to, an immunoglobulin fusion segment, an albumin fusion segment, and any other fusion segment that increases the biological half-life of the protein, provides flexibility to the protein, and/or enables multimerization. It is within the scope of the disclosure to use one or more fusion segments. Fusion segments can be joined to the amino terminus and/or carboxyl terminus of a sCTLA-4 protein of the embodiments. As used herein, join refers to combine by attachment using genetic engineering techniques. In such an embodiment, a sCTLA-4 protein can be joined directly to a fusion segment, or a sCTLA-4 protein can be linked to the fusion segment by a linker of one or more amino acids.

One embodiment is a sCTLA-4 fusion protein that comprises a sCTLA-4 protein and an immunoglobulin fusion segment. Examples of immunoglobulin fusion segments include one or more constant regions of an immunoglobulin, such as one or more constant regions of gamma, mu, alpha, delta or epsilon Ig heavy chains or of kappa or lambda Ig light chains. In one embodiment, an immunoglobulin fusion segment is at least one constant region of a gamma heavy chain. In one embodiment, an immunoglobulin fusion segment comprises the Fc region of an immunoglobulin. The Fc region of an IgG, IgA, or IgD antibody comprises the hinge and second and third constant regions (i.e., CH2 and CH3) of the respective antibody. The Fc region of an IgM antibody comprises the hinge and second, third and fourth constant regions (CH2, CH3 and CH4) of the respective antibody. In one embodiment, the immunoglobulin fusion segment comprises the Fc region of an IgG, such as IgG1. In one embodiment, the immunoglobulin fusion segment is an IgG Cγ1 (IgG C-gamma-1) segment. In one embodiment, the immunoglobulin fusion segment is a human IgG Cγ1 segment.

The disclosure also provides a sCTLA-4 protein that comprises a secretory segment (i.e., a secretory sequence) joined to the amino terminus of the sCTLA-4 protein. A secretory segment enables an expressed sCTLA-4 protein to be secreted from the cell that produces the protein. Suitable secretory segments include a CTLA-4 secretory segment or any heterologous secretory segment capable of directing the secretion of a sCTLA-4 protein, including a sCTLA-4 fusion protein, of the present invention. Examples of secretory segments include, but are not limited to, tissue plasminogen activator (t-PA), interferon, interleukin, growth hormone, histocompatibility and viral envelope glycoprotein secretory segments. In one embodiment, the secretory segment is an interleukin (IL) secretory segment. In one embodiment, the secretory segment is an IL6 secretory segment.

One embodiment of the disclosure is a sCTLA-4 protein comprising amino acid sequence SEQ ID NO:4. One embodiment is a sCTLA-4 protein that is at least 60%, at least 65%, at least 70%, at least 75%, at least 80%, at least 85%, at least 90%, or at least 95% identical to amino acid sequence SEQ ID NO:4. In one embodiment, a sCTLA-4 protein is at least 60% identical to amino acid sequence SEQ ID NO:4. In one embodiment, a sCTLA-4 protein is at least 65% identical to amino acid sequence SEQ ID NO:4. In one embodiment, a sCTLA-4 protein is at least 70% identical to amino acid sequence SEQ ID NO:4. In one embodiment, a sCTLA-4 protein is at least 75% identical to amino acid sequence SEQ ID NO:4. In one embodiment, a sCTLA-4 protein is at least 80% identical to amino acid sequence SEQ ID NO:4. In one embodiment, a sCTLA-4 protein is at least 85% identical to amino acid sequence SEQ ID NO:4. In one embodiment, a sCTLA-4 protein is at least 90% identical to amino acid sequence SEQ ID NO:4. In one embodiment, a sCTLA-4 protein is at least 95% identical to amino acid sequence SEQ ID NO:4. In each of these embodiments, the respective sCTLA-4 protein retains the ability to bind to a B7 protein.

One embodiment of the disclosure is a sCTLA-4 protein comprising amino acid sequence SEQ ID NO:5. One embodiment is a sCTLA-4 protein that is at least 60%, at least 65%, at least 70%, at least 75%, at least 80%, at least 85%, at least 90%, or at least 95% identical to amino acid sequence SEQ ID NO:5. In one embodiment, a sCTLA-4 protein is at least 60% identical to amino acid sequence SEQ ID NO:5. In one embodiment, a sCTLA-4 protein is at least 65% identical to amino acid sequence SEQ ID NO:5. In one embodiment, a sCTLA-4 protein is at least 70% identical to amino acid sequence SEQ ID NO:5. In one embodiment, a sCTLA-4 protein is at least 75% identical to amino acid sequence SEQ ID NO:5. In one embodiment, a sCTLA-4 protein is at least 80% identical to amino acid sequence SEQ ID NO:5. In one embodiment, a sCTLA-4 protein is at least 85% identical to amino acid sequence SEQ ID NO:5. In one embodiment, a sCTLA-4 protein is at least 90% identical to amino acid sequence SEQ ID NO:5. In one embodiment, a sCTLA-4 protein is at least 95% identical to amino acid sequence SEQ ID NO:5. In each of these embodiments, the respective sCTLA-4 protein retains the ability to bind to a B7 protein.

One embodiment is a sCTLA-4 fusion protein, wherein the sCTLA-4 domain of the fusion protein is at least 60%, at least 65%, at least 70%, at least 75%, at least 80%, at least 85%, at least 90%, or at least 95% identical to amino acid sequence SEQ ID NO:4. One embodiment is a sCTLA-4 fusion protein, wherein the sCTLA-4 domain of the fusion protein is at least 60% identical to amino acid sequence SEQ ID NO:4. One embodiment is a sCTLA-4 fusion protein, wherein the sCTLA-4 domain of the fusion protein is at least 65% identical to amino acid sequence SEQ ID NO:4. One embodiment is a sCTLA-4 fusion protein, wherein the sCTLA-4 domain of the fusion protein is at least 70% identical to amino acid sequence SEQ ID NO:4. One embodiment is a sCTLA-4 fusion protein, wherein the sCTLA-4 domain of the fusion protein is at least 75% identical to amino acid sequence SEQ ID NO:4. One embodiment is a sCTLA-4 fusion protein, wherein the sCTLA-4 domain of the fusion protein is at least 80% identical to amino acid sequence SEQ ID NO:4. One embodiment is a sCTLA-4 fusion protein, wherein the sCTLA-4 domain of the fusion protein is at least 85% identical to amino acid sequence SEQ ID NO:4. One embodiment is a sCTLA-4 fusion protein, wherein the sCTLA-4 domain of the fusion protein is at least 90% identical to amino acid sequence SEQ ID NO:4. One embodiment is a sCTLA-4 fusion protein, wherein the sCTLA-4 domain of the fusion protein is at least 95% identical to amino acid sequence SEQ ID NO:4. One embodiment is a sCTLA-4 fusion protein comprising a sCTLA-4 domain having amino acid SEQ ID NO:4 and a fusion segment, such as an immunoglobulin fusion segment. One embodiment is a sCTLA-4 fusion protein comprising a sCTLA-4 domain having amino acid SEQ ID NO:4 and an immunoglobulin fusion segment having the amino acid sequence encoded by the immunoglobulin fusion segment-encoding region of SEQ ID NO:1 (CTLA4IgG). In each of these embodiments, the respective sCTLA-4 protein retains the ability to bind to a B7 protein.

One embodiment is a sCTLA-4 fusion protein, wherein the sCTLA-4 domain of the fusion protein is at least 60%, at least 65%, at least 70%, at least 75%, at least 80%, at least 85%, at least 90%, or at least 95% identical to amino acid sequence SEQ ID NO:5. One embodiment is a sCTLA-4 fusion protein, wherein the sCTLA-4 domain of the fusion protein is at least 60% identical to amino acid sequence SEQ ID NO:5. One embodiment is a sCTLA-4 fusion protein, wherein the sCTLA-4 domain of the fusion protein is at least 65% identical to amino acid sequence SEQ ID NO:5. One embodiment is a sCTLA-4 fusion protein, wherein the sCTLA-4 domain of the fusion protein is at least 70% identical to amino acid sequence SEQ ID NO:5. One embodiment is a sCTLA-4 fusion protein, wherein the sCTLA-4 domain of the fusion protein is at least 75% identical to amino acid sequence SEQ ID NO:5. One embodiment is a sCTLA-4 fusion protein, wherein the sCTLA-4 domain of the fusion protein is at least 80% identical to amino acid sequence SEQ ID NO:5. One embodiment is a sCTLA-4 fusion protein, wherein the sCTLA-4 domain of the fusion protein is at least 85% identical to amino acid sequence SEQ ID NO:5. One embodiment is a sCTLA-4 fusion protein, wherein the sCTLA-4 domain of the fusion protein is at least 90% identical to amino acid sequence SEQ ID NO:5. One embodiment is a sCTLA-4 fusion protein, wherein the sCTLA-4 domain of the fusion protein is at least 95% identical to amino acid sequence SEQ ID NO:5. One embodiment is a sCTLA-4 fusion protein comprising a sCTLA-4 domain having amino acid SEQ ID NO:5 and a fusion segment, such as an immunoglobulin fusion segment. One embodiment is a sCTLA-4 fusion protein comprising a sCTLA-4 domain having amino acid SEQ ID NO:5 and an immunoglobulin fusion segment having the amino acid sequence encoded by the immunoglobulin fusion segment-encoding region of SEQ ID NO:1. In each of these embodiments, the respective sCTLA-4 protein retains the ability to bind to a B7 protein.

One embodiment is a sCTLA-4 protein comprising a secretory segment joined to a sCTLA-4 domain, wherein the sCTLA-4 domain comprises amino acid sequence SEQ ID NO:4. One embodiment is a sCTLA-4 protein comprising a secretory segment joined to a sCTLA-4 domain, wherein the sCTLA-4 domain is at least 60%, at least 65%, at least 70%, at least 75%, at least 80%, at least 85%, at least 90%, or at least 95% identical to amino acid sequence SEQ ID NO:4. One embodiment is a sCTLA-4 protein comprising a secretory segment joined to a sCTLA-4 domain joined to a fusion segment, wherein the sCTLA-4 domain comprises amino acid sequence SEQ ID NO:4. One embodiment is a sCTLA-4 protein comprising a secretory segment joined to a sCTLA-4 domain joined to a fusion segment, wherein the sCTLA-4 domain is at least 60%, at least 65%, at least 70%, at least 75%, at least 80%, at least 85%, at least 90%, or at least 95% identical to amino acid sequence SEQ ID NO:4.

One embodiment is a sCTLA-4 protein comprising a secretory segment joined to a sCTLA-4 domain, wherein the sCTLA-4 domain comprises amino acid sequence SEQ ID NO:5. One embodiment is a sCTLA-4 protein comprising a secretory segment joined to a sCTLA-4 domain, wherein the sCTLA-4 domain is at least 60%, at least 65%, at least 70%, at least 75%, at least 80%, at least 85%, at least 90%, or at least 95% identical to amino acid sequence SEQ ID NO:5. One embodiment is a sCTLA-4 protein comprising a secretory segment joined to a sCTLA-4 domain joined to a fusion segment, wherein the sCTLA-4 domain comprises amino acid sequence SEQ ID NO:5. One embodiment is a sCTLA-4 protein comprising a secretory segment joined to a sCTLA-4 domain joined to a fusion segment, wherein the sCTLA-4 domain is at least 60%, at least 65%, at least 70%, at least 75%, at least 80%, at least 85%, at least 90%, or at least 95% identical to amino acid sequence SEQ ID NO:5.

One embodiment of the disclosure is a sCTLA-4 protein having amino acid sequence SEQ ID NO:2. SEQ ID NO:2 represents a sCTLA-4 protein having an IL-6 secretory signal joined to a murine sCTLA-4 protein having SEQ ID NO:4. One embodiment is a sCTLA-4 protein that is at least 60%, at least 65%, at least 70%, at least 75%, at least 80%, at least 85%, at least 90%, or at least 95% identical to amino acid sequence SEQ ID NO:2. Such a sCTLA-4 protein optionally also includes a fusion segment of the embodiments. One embodiment is a sCTLA-4 fusion protein, the sCTLA-4 domain having amino acid sequence SEQ ID NO:2 and the immunoglobulin fusion segment having the amino acid sequence encoded by the immunoglobulin fusion segment-encoding region of SEQ ID NO:1.

### Nucleic Acids

The disclosure provides nucleic acid molecules that encode a sCTLA-4 protein of the embodiments. One embodiment is a nucleic acid molecule that encodes a sCTLA-4 protein that is not a fusion protein. One embodiment is a nucleic acid molecule that encodes a sCTLA-4 fusion protein. One embodiment is a nucleic acid molecule that encodes a sCTLA-4 protein that has a secretory segment at its amino terminus, such as a sCTLA-4 fusion protein joined to a secretory segment.

In one embodiment, a nucleic acid molecule encodes a sCTLA-4 protein comprising amino acid sequence SEQ ID NO:4. One embodiment is a nucleic acid molecule that encodes a sCTLA-4 protein that is at least 70%, at least 75%, at least 80%, at least 85%, at least 90%, or at least 95% identical to amino acid sequence SEQ ID NO:4. In one embodiment, a nucleic acid molecule encodes a sCTLA-4 protein that is at least 70% identical to amino acid sequence SEQ ID NO:4. In one embodiment, a nucleic acid molecule encodes a sCTLA-4 protein that is at least 75% identical to amino acid sequence SEQ ID NO:4. In one embodiment, a nucleic acid molecule encodes a sCTLA-4 protein that is at least 80% identical to amino acid sequence SEQ ID NO:4. In one embodiment, a nucleic acid molecule encodes a sCTLA-4 protein that is at least 85% identical to amino acid sequence SEQ ID NO:4. In one embodiment, a nucleic acid molecule encodes a sCTLA-4 protein that is at least 90% identical to amino acid sequence SEQ ID NO:4. In one embodiment, a sCTLA-4 protein is at least 95% identical to amino acid sequence SEQ ID NO:4. In each of these embodiments, the sCTLA-4 protein encoded by the respective nucleic acid molecule retains the ability to bind to a B7 protein.

In one embodiment, a nucleic acid molecule encodes a sCTLA-4 protein comprising amino acid sequence SEQ ID NO:5. One embodiment is a nucleic acid molecule that encodes a sCTLA-4 protein that is at least 70%, at least 75%, at least 80%, at least 85%, at least 90%, or at least 95% identical to amino acid sequence SEQ ID NO:5. In one embodiment, a nucleic acid molecule encodes a sCTLA-4 protein that is at least 70% identical to amino acid sequence SEQ ID NO:5. In one embodiment, a nucleic acid molecule encodes a sCTLA-4 protein that is at least 75% identical to amino acid sequence SEQ ID NO:5. In one embodiment, a nucleic acid molecule encodes a sCTLA-4 protein that is at least 80% identical to amino acid sequence SEQ ID NO:5. In one embodiment, a nucleic acid molecule encodes a sCTLA-4 protein that is at least 85% identical to amino acid sequence SEQ ID NO:5. In one embodiment, a nucleic acid molecule encodes a sCTLA-4 protein that is at least 90% identical to amino acid sequence SEQ ID NO:5. In one embodiment, a sCTLA-4 protein is at least 95% identical to amino acid sequence SEQ ID NO:5. In each of these embodiments, the sCTLA-4 protein encoded by the respective nucleic acid molecule retains the ability to bind to a B7 protein.

In one embodiment, a nucleic acid molecule comprises nucleic acid sequence SEQ ID NO:3. One embodiment is a nucleic acid molecule that is at least 70%, at least 75%, at least 80%, at least 85%, at least 90%, or at least 95% identical to nucleic acid sequence SEQ ID NO:3. One embodiment is a nucleic acid molecule that is at least 70% identical to nucleic acid sequence SEQ ID NO:3. One embodiment is a nucleic acid molecule that is at least 75% identical to nucleic acid sequence SEQ ID NO:3. One embodiment is a nucleic acid molecule that is at least 80% identical to nucleic acid sequence SEQ ID NO:3. One embodiment is a nucleic acid molecule that is at least 85% identical to nucleic acid sequence SEQ ID NO:3. One embodiment is a nucleic acid molecule that is at least 90% identical to nucleic acid sequence SEQ ID NO:3. One embodiment is a nucleic acid molecule that is at least 95% identical to nucleic acid sequence SEQ ID NO:3. In each of these embodiments, the sCTLA-4 protein encoded by the respective nucleic acid molecule retains the ability to bind to a B7 protein.

One embodiment is a nucleic acid molecule that encodes a sCTLA-4 fusion protein, wherein the sCTLA-4 protein domain is at least 70%, at least 75%, at least 80%, at least 85%, at least 90%, or at least 95% identical to amino acid sequence SEQ ID NO:4. One embodiment is a nucleic acid molecule that encodes a sCTLA-4 fusion protein, wherein the sCTLA-4 protein domain comprises amino acid SEQ ID NO:4. In each of these embodiments, the sCTLA-4 fusion protein encoded by the respective nucleic acid molecule retains the ability to bind to a B7 protein.

One embodiment is a nucleic acid molecule that encodes a sCTLA-4 fusion protein, wherein the sCTLA-4 protein domain is at least 70%, at least 75%, at least 80%, at least 85%, at least 90%, or at least 95% identical to amino acid sequence SEQ ID NO:5. One embodiment is a nucleic acid molecule that encodes a sCTLA-4 fusion protein, wherein the sCTLA-4 protein domain comprises amino acid SEQ ID NO:5. In each of these embodiments, the sCTLA-4 fusion protein encoded by the respective nucleic acid molecule retains the ability to bind to a B7 protein.

One embodiment is a nucleic acid molecule that encodes a sCTLA-4 fusion protein, wherein the sCTLA-4 protein domain is encoded by a nucleic acid molecule that is at least 70%, at least 75%, at least 80%, at least 85%, at least 90%, at least 95%, or at least 100% identical to nucleic acid sequence SEQ ID NO:3. In each of these embodiments, the sCTLA-4 fusion protein encoded by the respective nucleic acid molecule retains the ability to bind to a B7 protein.

One embodiment is a nucleic acid molecule that encodes a sCTLA-4 protein comprising a secretory segment joined to a sCTLA-4 domain, wherein the sCTLA-4 domain comprises amino acid sequence SEQ ID NO:4. One embodiment is a nucleic acid molecule that encodes a sCTLA-4 protein comprising a secretory segment joined to a sCTLA-4 domain, wherein the sCTLA-4 domain is at least 60%, at least 65%, at least 70%, at least 75%, at least 80%, at least 85%, at least 90%, or at least 95% identical to amino acid sequence SEQ ID NO:4. One embodiment is a nucleic acid molecule that encodes a sCTLA-4 fusion protein comprising a secretory segment joined to a sCTLA-4 domain, wherein the sCTLA-4 domain comprises amino acid sequence SEQ ID NO:4. One embodiment is a nucleic acid molecule that encodes a sCTLA-4 fusion protein comprising a secretory segment joined to a sCTLA-4 domain, wherein the sCTLA-4 domain is at least 60%, at least 65%, at least 70%, at least 75%, at least 80%, at least 85%, at least 90%, or at least 95% identical to amino acid sequence SEQ ID NO:4.

One embodiment is a nucleic acid molecule that encodes a sCTLA-4 protein comprising a secretory segment joined to a sCTLA-4 domain, wherein the sCTLA-4 domain comprises amino acid sequence SEQ ID NO:5. One embodiment is a nucleic acid molecule that encodes a sCTLA-4 protein comprising a secretory segment joined to a sCTLA-4 domain, wherein the sCTLA-4 domain is at least 60%, at least 65%, at least 70%, at least 75%, at least 80%, at least 85%, at least 90%, or at least 95% identical to amino acid sequence SEQ ID NO:5. One embodiment is a nucleic acid molecule that encodes a sCTLA-4 fusion protein comprising a secretory segment joined to a sCTLA-4 domain, wherein the sCTLA-4 domain comprises amino acid sequence SEQ ID NO:5. One embodiment is a nucleic acid molecule that encodes a sCTLA-4 fusion protein comprising a secretory segment joined to a sCTLA-4 domain, wherein the sCTLA-4 domain is at least 60%, at least 65%, at least 70%, at least 75%, at least 80%, at least 85%, at least 90%, or at least 95% identical to amino acid sequence SEQ ID NO:5.

One embodiment is a nucleic acid molecule that encodes a sCTLA4 protein comprising a secretory segment joined to a sCTLA-4 domain, wherein the sCTLA-4 encoding domain comprises SEQ ID NO:3. One embodiment is a nucleic acid molecule that encodes a sCTLA4 protein comprising a secretory segment joined to a sCTLA-4 domain, wherein the sCTLA-4 encoding domain is at least 60%, at least 65%, at least 70%, at least 75%, at least 80%, at least 85%, at least 90%, or at least 95% identical to nucleic acid sequence SEQ ID NO:3. One embodiment is a nucleic acid molecule that encodes a sCTLA4 fusion protein comprising a secretory segment joined to a sCTLA-4 domain, wherein the sCTLA-4 encoding domain comprises SEQ ID NO:3. One embodiment is a nucleic acid molecule that encodes a sCTLA4 fusion protein comprising a secretory segment joined to a sCTLA-4 domain, wherein the sCTLA-4 encoding domain is at least 60%, at least 65%, at least 70%, at least 75%, at least 80%, at least 85%, at least 90%, or at least 95% identical to nucleic acid sequence SEQ ID NO:3.

One embodiment of the disclosure is a nucleic acid molecule that encodes a sCTLA-4 protein having amino acid sequence SEQ ID NO:2. One embodiment is a nucleic acid molecule that encodes a sCTLA-4 protein that is at least 60%, at least 65%, at least 70%, at least 75%, at least 80%, at least 85%, at least 90%, or at least 95% identical to amino acid sequence SEQ ID NO:2. Such a sCTLA-4 protein optionally also includes a fusion segment of the embodiments. One embodiment is a nucleic acid molecule that encodes a sCTLA-4 fusion protein, the sCTLA-4 domain having amino acid sequence SEQ ID NO:2 and the immunoglobulin fusion segment having the amino acid sequence encoded by the immunoglobulin fusion segment-encoding region of SEQ ID NO:1.

### Vectors and Virions

Adeno-associated virus (AAV) is a unique, non-pathogenic member of the Parvoviridae family of small, non-enveloped, single-stranded DNA animal viruses. AAV require helper virus (e.g., adenovirus) for replication and, thus, do not replicate upon administration to a subject. AAV can infect a relatively wide range of cell types and stimulate only a mild immune response, particularly as compared to a number of other viruses, such as adenovirus. A number of AAV serotypes have been identified. Examples include AAV1, AAV2, AAV3, AAV4, AAV5, AAV6, AAV7, AAV8, AAV9, AAV10, AAV11, and AAV12, which appear to be of simian or human origin. AAV have also been found in other animals, including birds (e.g., avian AAV, or AAAV), bovines (e.g., bovine AAV, or BAAV), canines, equines, ovines, and porcines.

AAV vectors are recombinant nucleic acid molecules in which at least a portion of the AAV genome is replaced by a heterologous nucleic acid molecule. It is possible to replace about 4.7 kilobases (kb) of AAV genome DNA, e.g., by removing the viral replication and capsid genes. Often the heterologous nucleic acid molecule is simply flanked by AAV inverted terminal repeats (ITRs) on each terminus. The ITRs serve as origins of replication and contain cis acting elements required for rescue, integration, excision from cloning vectors, and packaging. Such vectors typically also include a promoter operatively linked to the heterologous nucleic acid molecule to control expression.

An AAV vector can be packaged into an AAV capsid in vitro with the assistance of a helper virus or helper functions expressed in cells to yield an AAV virion. The serotype and cell tropism of an AAV virion are conferred by the nature of the viral capsid proteins.

AAV vectors and AAV virions have been shown to transduce cells efficiently, including both dividing and non-dividing cells. AAV vectors and virions have been shown to be safe and to lead to long term in vivo persistence and expression in a variety of cell types.

As used herein, an AAV vector that encodes a sCTLA-4 protein is a nucleic acid molecule that comprises a nucleic acid molecule that encodes a sCTLA-4 protein of the embodiments, an ITR joined to 5′ terminus of the sCTLA-4 nucleic acid molecule, and an ITR joined to the 3′ terminus of the sCTLA-4 nucleic acid molecule. Examples of ITRs include, but are not limited, to AAV1, AAV2, AAV3, AAV4, AAV5, AAV6, AAV7, AAV8, AAV9, AAV10, AAV11, AAV12, AAAV, BAAV, and other AAV ITRs known to those skilled in the art. In one embodiment, an AAV ITR is selected from an AAV2 ITR, an AAV5 ITR, an AAV6 ITR, and a BAAV ITR. In one embodiment, an AAV ITR is an AAV2 ITR. In one embodiment, an AAV ITR is an AAV5 ITR. In one embodiment, an AAV ITR is an AAV6 ITR. In one embodiment, an AAV ITR is a BAAV ITR.

An AAV vector of the embodiments can also include other sequences, such as expression control sequences. Examples of expression control sequences include, but are not limited to, a promoter, an enhancer, a repressor, a ribosome binding site, an RNA splice site, a polyadenylation site, a transcriptional terminator sequence, and a microRNA binding site. Examples of promoters include, but are not limited to, an AAV promoter, such as a p5, p19 or p40 promoter, an adenovirus promoter, such as an adenoviral major later promoter, a cytomegalovirus (CMV) promoter, a papilloma virus promoter, a polyoma virus promoter, a respiratory syncytial virus (RSV) promoter, a sarcoma virus promoter, an SV40 promoter, other viral promoters, an actin promoter, an amylase promoter, an immunoglobulin promoter, a kallikrein promoter, a metallothionein promoter, a heat shock promoter, an endogenous promoter, a promoter regulated by rapamycin or other small molecules, other cellular promoters, and other promoters known to those skilled in the art. In one embodiment, the promoter is an AAV promoter. In one embodiment, the promoter is a CMV promoter. Selection of expression control sequences to include can be accomplished by one skilled in the art.

The disclosure provides AAV vectors of different serotypes (as determined by the serotype of the ITRs within such vector) that encode a sCTLA-4 protein of the embodiments. Such an AAV vector can be selected from an AAV1 vector, an AAV2 vector, an AAV3 vector, an AAV4 vector, an AAV5 vector, an AAV6 vector, an AAV7 vector, an AAV8 vector, an AAV9 vector, an AAV10 vector, an AAV11 vector, an AAV 12 vector, an AAAV vector, and a BAAV vector, wherein any of such vectors encode a sCTLA-4 protein of the embodiments. One embodiment is an AAV2 vector, an AAV5 vector, an AAV6 vector or a BAAV vector, wherein the respective vector encodes a sCTLA-4 protein of the embodiments. One embodiment is an AAV2 vector that encodes a sCTLA-4 protein of the embodiments. One embodiment is an AAV5 vector that encodes a sCTLA-4 protein of the embodiments. One embodiment is an AAV6 vector that encodes a sCTLA-4 protein of the embodiments. One embodiment is a BAAV vector that encodes a sCTLA-4 protein of the embodiments.

One embodiment is an AAV vector that comprises AAV ITRs and a CMV promoter operatively linked to a nucleic acid molecule encoding a sCTLA-4 protein of the embodiments. One embodiment is an AAV vector that comprises AAV ITRs and a CMV promoter operatively linked to a nucleic acid molecule encoding a sCTLA-4 fusion protein of the embodiments. One embodiment is an AAV2 vector that comprises AAV2 ITRs and a CMV promoter operatively linked to a nucleic acid molecule encoding a sCTLA-4 protein of the embodiments. One embodiment is an AAV2 vector that comprises AAV2 ITRs and a CMV promoter operatively linked to a nucleic acid molecule encoding a sCTLA-4 fusion protein of the embodiments. One embodiment is an AAV2 vector that comprises AAV2 ITRs and a CMV promoter operatively linked to a nucleic acid molecule encoding a sCTLA-4-IgG fusion protein of the embodiments.

One embodiment is an AAV vector that has nucleic acid sequence SEQ ID NO:1.

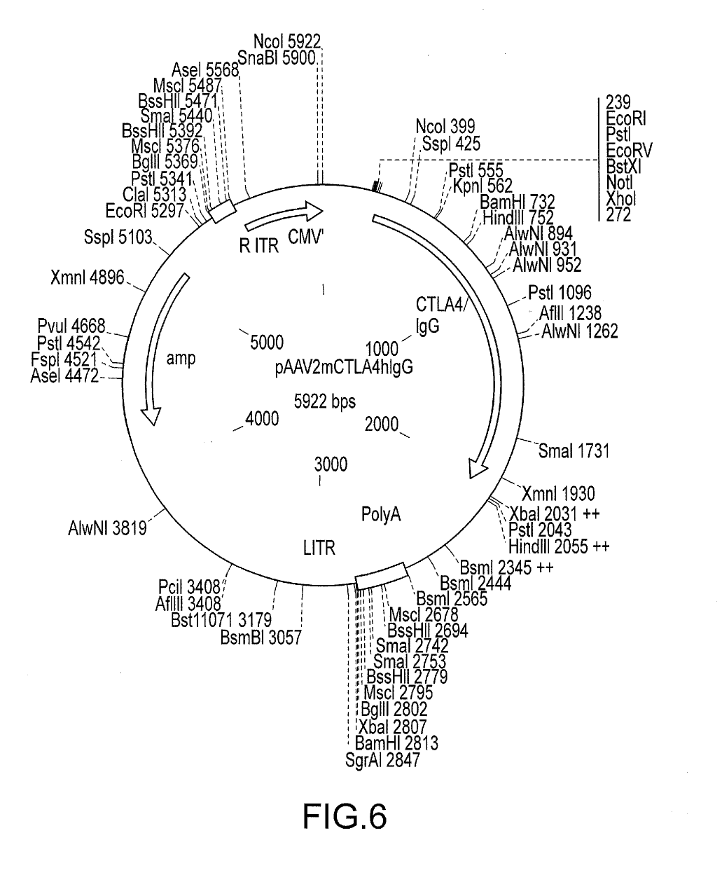

The disclosure provides plasmid vectors that encode a sCTLA-4 protein of the embodiments. Such plasmid vectors also include control regions, such as AAV ITRs, a promoter operatively linked to the nucleic acid molecule encoding the sCTLA-4 protein, one or more splice sites, a polyadenylation site, and a transcription termination site. Such plasmid vectors also typically include a number of restriction enzyme sites as well as a nucleic acid molecule that encodes drug resistance. An example of a plasmid vector is pAAV2-CMV-mCTLA4-hIgG (SEQ ID NO:1), a schematic of which is shown in FIG. 6.

The disclosure provides an AAV virion. An AAV virion is an AAV vector encoding a sCTLA-4 protein of the embodiments encapsidated in an AAV capsid. Examples of AAV capsids include AAV1 capsids, AAV2 capsids, AAV3 capsids, AAV4 capsids, AAV5 capsids, AAV6 capsids, AAV7 capsids, AAV8 capsids, AAV9 capsids, AAV10 capsids, AAV 11 capsids, AAV12 capsids, AAAV capsids, BAAV capsids, and capsids from other AAV serotypes known to those skilled in the art. In one embodiment, the capsid is a chimeric capsid, i.e., a capsid comprising VP proteins from more than one serotype. As used herein, the serotype of an AAV virion of the embodiments is the serotype conferred by the VP capsid proteins. For example, an AAV2 virion is a virion comprising AAV2 VP1, VP2 and VP3 proteins.

One embodiment of the disclosure is an AAV virion selected from an AAV2 virion, an AAV5 virion, an AAV6 virion, and a BAAV virion, wherein the AAV vector within the virion encodes a sCTLA-4 protein of the embodiments. One embodiment is an AAV2 virion, wherein the AAV vector within the virion encodes a sCTLA-4 protein of the embodiments. One embodiment is an AAV5 virion, wherein the AAV vector within the virion encodes a sCTLA-4 protein of the embodiments. One embodiment is an AAV6 virion, wherein the AAV vector within the virion encodes a sCTLA-4 protein of the embodiments. One embodiment is a BAAV virion, wherein the AAV vector within the virion encodes a sCTLA-4 protein of the embodiments.

One embodiment is an AAV virion that comprises an AAV vector that has nucleic acid sequence SEQ ID NO:1.

Methods useful for producing AAV vectors and AAV virions disclosed herein are known to those skilled in the art and are also exemplified in the Examples. Briefly, an AAV vector of the embodiments can be produced using recombinant DNA or RNA techniques to isolate nucleic acid sequences of interest and join them together as described herein, e.g., by using techniques known to those skilled in the art, such as restriction enzyme digestion, ligation, PCR amplification, and the like. Methods to produce an AAV virion of the embodiments typically include (a) introducing an AAV vector of the embodiments into a host, (b) introducing a helper vector into the host cell, wherein the helper vector comprises the viral functions missing from the AAV vector and (c) introducing a helper virus into the host cell. All functions for AAV virion replication and packaging need to be present, to achieve replication and packaging of the AAV vector into AAV virions. In some instances, at least one of the viral functions encoded by the helper vector can be expressed by the host cell. Introduction of the vectors and helper virus can be carried out using standard techniques and occur simultaneously or sequentially. The host cells are then cultured to produce AAV virions, which are then purified using standard techniques, such as CsCl gradients. Residual helper virus activity can be inactivated using known methods, such as heat inactivation. Such methods typically result in high titers of highly purified AAV virions that are ready for use. In some embodiments, an AAV vector of a specified serotype is packaged in a capsid of the same serotype. For example, an AAV2 vector can be packaged in an AAV2 capsid. In other embodiments, an AAV vector of a specified serotype is packaged in a capsid of a different serotype in order to modify the tropism of the resultant virion. Combinations of AAV vector serotypes and AAV capsid serotypes can be determined by those skilled in the art.

### Composition and Method of Use

The disclosure provides a composition comprising an AAV vector encoding a sCTLA-4 protein of the embodiments. The disclosure also provides a composition comprising an AAV virion comprising an AAV vector encoding a sCTLA-4 protein of the embodiments. Such compositions can also include an aqueous solution, such as a physiologically compatible buffer. Examples of excipients include water, saline, Ringer's solution, and other aqueous physiologically balanced salt solutions. In some embodiments, excipients are added to, for example, maintain particle stability or to prevent aggregation. Examples of such excipients include, but are not limited to, magnesium to maintain particle stability, pluronic acid to reduce sticking, mannitol to reduce aggregation, and the like, known to those skilled in the art.

A composition of the embodiments is conveniently formulated in a form suitable for administration to a subject. Techniques to formulate such compositions are known to those skilled in the art. For example, an AAV virion of the embodiments can be combined with saline or other pharmaceutically acceptable solution; in some embodiments excipients are also added. In another embodiment, a composition comprising an AAV virion is dried, and a saline solution or other pharmaceutically acceptable solution can be added to the composition prior to administration.

The disclosure provides a method to protect a subject from Sjögren's syndrome. Such a method includes the step of administering to the subject an AAV virion comprising an AAV vector that encodes a sCTLA-4 protein of the embodiments. As used herein, the ability of an AAV virion of the embodiments to protect a subject from Sjögren's syndrome refers to the ability of such AAV virion to prevent, treat, or ameliorate symptoms of Sjögren's syndrome. In one embodiment, an AAV virion of the embodiments prevents symptoms of Sjögren's syndrome. In one embodiment, an AAV virion of the embodiments treats symptoms of Sjögren's syndrome. In one embodiment, an AAV virion of the embodiments ameliorates symptoms of Sjögren's syndrome. In one embodiment, an AAV virion of the embodiments prevents symptoms of Sjögren's syndrome from occurring in a subject, for example in a subject susceptible to Sjögren's syndrome. In one embodiment, an AAV virion of the embodiments prevents symptoms of Sjögren's syndrome from worsening. In one embodiment, an AAV virion of the embodiments reduces symptoms of Sjögren's syndrome in a subject. In one embodiment, an AAV virion of the embodiments enables a subject to recover from symptoms of Sjögren's syndrome. Sjögren's syndrome can lead to a number of symptoms including, but not limited to the following: reduced salivary function, which can result in xerostomia (dry mouth); reduced lachrymal gland function, which can result in xerophthalmia (conjunctivitis sicca, dry eyes); immune cell infiltration (e.g., T cells, B cells, macrophages) of salivary glands; immune cell infiltration of lachrymal glands; increase in proinflammatory cytokines (e.g., Th1-cell cytokines, Th17-cell cytokines); decrease in nTreg cytokines, increase in circulating autoantibodies such as antinuclear antibodies (ANA), SSA antibodies (e.g., SSA/Ro), SSB antibodies (e.g., SSB/La), and M3R antibodies; and fatigue. Methods to measure such symptoms are known to those skilled in the art and are described in the Examples.

The disclosure provides a method comprising administering an AAV virion comprising an AAV vector that encodes a sCTLA-4 protein of the embodiments to a subject, wherein such administration maintains salivary gland function in such a subject. As used herein, maintaining salivary gland function means that salivary gland function after administration of an AAV virion of the embodiments to a subject is equivalent to salivary gland function in that subject prior to administration of the AAV virion; for example, in the case of a subject with normal salivary gland function, the function remains normal after AAV virion administration; if the subject has symptoms, the salivary gland function does not worsen after administration of the AAV virion, but is equivalent to function prior to AAV virion administration. Also provided is a method comprising administering AAV virion comprising an AAV vector that encodes a sCTLA-4 protein of the embodiments to a subject, wherein such administration improves salivary gland function in such a subject. The disclosure provides a method comprising administering an AAV virion comprising an AAV vector that encodes a sCTLA-4 protein of the embodiments to a subject, wherein such administration maintains lachrymal gland function in such a subject. Also provided is a method comprising administering an AAV virion comprising an AAV vector that encodes a sCTLA-4 protein of the embodiments to a subject, wherein such administration improves lachrymal gland function in such a subject.

The disclosure provides a method comprising administering an AAV virion comprising an AAV vector that encodes a sCTLA-4 protein of the embodiments to a subject with Sjögren's syndrome, wherein such administration reduces immune cell infiltration in salivary glands of such a subject. The disclosure provides a method comprising administering an AAV virion comprising an AAV vector that encodes a sCTLA-4 protein of the embodiments to a subject with Sjögren's syndrome, wherein such administration reduces immune cell infiltration in lachrymal glands of such a subject. Examples of immune cells that infiltrate glands of subjects with Sjögren's syndrome include B cells, T cells, and macrophages.

As used herein, a subject is any animal that is susceptible to Sjögren's syndrome. Subjects include humans and other mammals, such as cats, dogs, horses, other companion animals, other zoo animals, lab animals (e.g., mice), and livestock.

An AAV virion of the embodiments can be administered in a variety of ways, such as by oral, intranasal, intraocular, conjunctival, intravenous, intraperitoneal, intramuscular, subcutaneous, intradermal, transdermal, topical, and rectal administration routes. In some embodiments, an AAV virion is administered by aerosol. In some embodiments, an AAV virion is administered to the mucosa. In some embodiments, an AAV virion is administered directly to a tissue or organ. In some embodiments, an AAV virion of the embodiments is administered to a salivary gland. In some embodiments, an AAV virion of the embodiments is administered to a lachrymal gland. In some embodiments, an AAV virion of the embodiments is administered to the lung, for example, by inhalation. In some embodiments, an AAV virion of the embodiments is administered to the kidney.

The disclosure provides for a method to protect a subject from Sjögren's syndrome in which an AAV virion of the embodiments is administered to a salivary gland of the subject. It was surprising that this administration route led to protection from Sjögren's syndrome in view of the unpredictability of protein sorting in the salivary gland; see, for example, Voutetakis et al., 2008, Hum Gene Ther 19, 1401-1405, and Perez et al., 2010, Int J Biochem Cell Biol 42, 773-777, Epub 2010 Feb. 26. In one embodiment an AAV2 virion of the embodiments is administered to a salivary gland. Such administration can occur, for example, by cannulation, e.g., retrograde cannulation.

The disclosure also provides a method to protect a subject from Sjögren's syndrome in which an AAV virion of the embodiments is administered to a lachrymal gland of the subject. In one embodiment, an AAV5 virion of the embodiments is administered to a lachrymal gland.

The disclosure also provides ex vivo methods to protect a subject from Sjögren's syndrome. Such methods can involve administering an AAV virion of the embodiments to a cell, tissue, or organ outside the body of the subject, and then placing that cell, tissue, or organ into the body. Such methods are known to those skilled in the art.

The dose of compositions disclosed herein to be administered to a subject to be effective (i.e., to protect a subject from Sjögren's syndrome) will depend on the subject's condition, manner of administration, and judgment of the prescribing physician. Often a single dose can be sufficient; however, the dose can be repeated if desirable. In general, the dose can range from about 10⁸ virion particles per kilogram to about 10¹² virion particles per kilogram.

The disclosure provides a treatment for Sjögren's syndrome. Such a treatment comprises an AAV virion comprising an AAV vector that encodes a sCTLA-4 protein. Administration of such a treatment to a subject protects the subject from Sjögren's syndrome.

The disclosure also provides a preventative for Sjögren's syndrome. Such a preventative comprises an AAV virion comprising an AAV vector that encodes a sCTLA-4 protein. Administration of such a preventative to a subject protects the subject from Sjögren's syndrome.

The disclosure provides a salivary gland cell transfected with an AAV vector that encodes a sCTLA-4 protein. The salivary gland cell can be that of a subject with Sjögren's syndrome. In one embodiment, the salivary gland cell is that of a subject with Sjögren's syndrome.

The disclosure provides an AAV virion comprising an AAV vector that encodes a sCTLA-4 protein of the embodiments for the treatment or prevention of Sjögren's syndrome. In one embodiment, such an AAV virion is useful for protecting a subject from Sjögren's syndrome. In one embodiment, such an AAV virion is useful for treating a subject with Sjögren's syndrome. In one embodiment, such an AAV virion is useful for preventing Sjögren's syndrome in a subject. The disclosure also provides for the use of an AAV virion comprising an AAV vector that encodes a sCTLA-4 protein of the embodiments for the preparation of a medicament to protect a subject from Sjögren's syndrome.

### EXAMPLES

The following examples are put forth so as to provide those of ordinary skill in the art with a complete disclosure and description of how to make and use the embodiments, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Efforts have also been made to ensure accuracy with respect to nucleic acid sequences and amino acid sequences presented, but some experimental errors and deviations should be accounted for. Unless indicated otherwise, parts are parts by weight, molecular weight is weight average molecular weight, and temperature is in degrees Celsius. Standard abbreviations are used.

### Example 1

### Materials and Methods

### Cell Lines

HEK-293 T cells were grown in Dulbecco's modified Eagle's medium (DMEM). Medium was supplemented with 10% heat-inactivated fetal bovine serum (Life Technologies, Rockville, Md., USA), 2 mM L-glutamine, penicillin (100 U/ml), and streptomycin (100 μg/ml; Biofluids, Rockville, Md., USA) as previously described (Kok et al., 2003, Hum Gene Ther 14, 1605-1618).

### Production of Virion AAV2-LacZ

Virion AAV2-LacZ encoding β-galactosidase was produced as described in Kaludov et al., 2001, J Virol 75, 6884-6993.

### Expression of a CTLA4IgG Protein In Vitro

Plasmid vector pAAV2-CMV-mCTLA4-hIgG (SEQ ID NO:1) was transfected into 293 cells, and secretion of the encoded proteins in the supernatant was determined by western blotting by using anti-mCTLA4 Antibody (R&D Systems, Minneapolis, Minn., USA).

### Competitive Inhibition of B7 Association by CTLA4IgG In Vitro

Mouse macrophages (CRL-2751, ATCC) were grown in DMEM with 4 mM L-glutamine, 1.5 g/L sodium bicarbonate, 4.5 g/L glucose (Biofluids, Rockville, Md., USA), 10% fetal bovine serum, and 20% LADMAC conditioned Media (produced from the LADMAC cell line (CRL-2420) at 37° C. in a humidified, 5% CO₂ atmosphere, incubator. 1×10⁵ cells/well were placed in round bottom 96-well plates and span down at 1500 rpm in a bench top centrifuge at 4° C. The cells were then washed twice with PBS (pH 7.4, 0.05% Tween 20), and incubated for 1 h at 37° C. with either medium from native HEK-293 cells or from HEK-293 cells transfected with AAV2-CTLA4IgG. Following additional wash, the cells were incubated in the dark with 0.5-1 ug/ml of Armenian hamster IgG FITC-conjugated anti B7-1 (Santa Cruz Biotechnology, Santa Cruz, Calif., USA) in blocking solution (PBS, pH 7.4, 0.5% BSA) at 4° C. for 40 min. The cells were then washed and analyzed with FACS.

### Animals

The C57BL16.NOD-Aec1Aec2 mouse model for Sjögren's syndrome is derived from the NOD mouse and mimics the pathophysiological characteristics of the disease with reduced salivary and lachrymal gland function, but lacks type I diabetes associated with the NOD mice (Cha et al., 2002, Arthritis and Rheumatism 46, 1390-1398). Further immunological characterization in the salivary and lachrymal glands indicated infiltrates of CD4 T cell, especially Th17. Moreover these mice also express elevated levels of proinflammatory cytokines as well as autoantibodies such as antinuclear antibodies (ANA) and M3R(Nguyen et al., ibid.).

Three female and ten male 6-week old C57BL/6.NOD-Aec1Aec2 mice were bred and maintained at the animal facility of the Department of Pathology, University of Florida, as described previously (Cha et al., ibid.). Baseline saliva and tear flow were collected from these mice when they were 6 weeks old. Gene therapy studies in C57BL/6.NOD-Aec1Aec2 mice, as described herein, were approved by the University of Florida the University of Florida's IACUC and IBC.

### Administration of Recombinant AAV2 Virions

Mice were randomly grouped, and AAV2 virions encoding CTLA4IgG or beta-galactosidase were delivered into the submandibular glands by retrograde instillation as previously described (Kok et al., ibid.) (AAV2-LacZ: 1 female, 5 males and AAV2-CTLA4IgG: 2 females, 5 males). The AAV2 virions were well tolerated; the mice showed no virion-related inflammation. Briefly, mild anesthesia was induced in eight-week old mice by ketamine (100 mg/mL, 1 mL/kg body weight; Fort Dodge Animal Health, Fort Dodge, Iowa, USA) and xylazine (20 mg/mL, 0.7 mL/kg body weight; Phoenix Scientific, St. Joseph, Mo., USA) solution given intramuscularly (IM). Ten minutes after IM injection of atropine (0.5 mg/kg BW; Sigma, St. Louis, Mo., USA), mice at the age of 8 weeks were administered 50 μl virion into both submandibular glands by retrograde ductal instillation (1×10¹° particles/gland) using a thin cannula.

Detection of CTLA4IgG Expression in Salivary Glands and Serum from C57BL/6.NOD-Aec1Aec2 Mice

To confirm the stable expression of CTLA4IgG in vivo after local delivery in the salivary glands from C57BL/6.NOD-Aec1Aec2 mice, homogenates of salivary glands were prepared as described previously (Vosters et al., ibid.). Briefly after measuring the wet weight, salivary glands were homogenized in protease buffer (phosphate buffered saline (PBS, Invitrogen, Carlsbad, Calif.)/0.05% Tween and complete protease inhibitor cocktail (Roche Applied Science, Indianapolis, Ind., USA). The excessive connective tissue and large aggregate debris was removed by 15 minutes centrifugation at 1500×g and the total protein in the supernatant was determined with BCA™ protein assay kit (Pierce, Rockford, Ill., USA) according to the manufacturer's instructions.

Serum collection was done at the time of sacrificing: Blood was collected by cardiac puncture and collected in microcentrifuge tubes. Serum was separated by centrifugation for 20 min at 2000 g and stored at −80° C.

For developing a sandwich-ELISA to determinate chimera of mouse CTLA4 and human IgG (mCTLA4/hIgG), a 96-well plate (Nunc, Rochester, N.Y., USA) was incubated overnight with 0.4 μg/mL capture antibody, goat anti-mouse CTLA-4 antibody (R&D Systems, Minneapolis, Minn., USA) in carbonate/bicarbonate buffer (pH 9.5). The next day, wells were washed with PBS and blocked with 5% normal goat serum/PBS for 2 hr at room temperature (RT). Fluid was discarded and incubated with 1004 of appropriately diluted standard control (0.0850 ug/mL rmCTLA4, R&D Systems, Minneapolis, Minn., USA) according to the product instruction or salivary gland homogenates in blocking buffer for 2 hr at RT. The wells were washed three times with PBS/0.05% Tween and incubated with 1:5000 dilution of detection antibody peroxidase affinity purified goat anti-human IgG (Jackson ImmunoResearch, West Grove, Pa.) for 1 hr at RT. Thereafter, the wells were washed 3 times, incubated with substrate (0.11M sodium-acetate buffer pH 5.5, 3% H₂O₂ and 10 mg/ml TMB in DMSO, R&D Systems, Minneapolis, Minn., USA) for 20 minutes in the dark at RT. The reaction was stopped by 1M H₂SO₄. The OD was measured at 450 nm using a Microplate reader model 680 (Bio-Rad Laboratories, Hercules, Calif., USA).

### Measurement of Salivary and Tear Flow Rates

Saliva collection was done as described previously (Nguyen et al., ibid.) at several time points: baseline (6 wks of age, 2 weeks before cannulation), 12, 16, 22, 26 and 30 weeks of age. Briefly, to measure stimulated flow rates of saliva (SFR), individual non-anesthetized mice were weighed and given an intraperitoneal (i.p.) injection of 100 μl of PBS containing isoproterenol (0.02 mg/ml) and pilocarpine (0.05 mg/ml). Saliva was collected from the oral cavity of individual mice for 10 min using a micropipette starting 1 min after the injection of the secretagogue. The volume of saliva sample was measured. To test stimulated flow rates of tears (TFR), individual mice were injected with pilocarpine hydrochloride (4.5 mg/kg in PBS) and allowed to rest comfortably for 10 min. SFR and TFR were calculated per gram body weight.

At week 30, tear volumes from individual animals were determined, after i.p. injection of pilocarpine (4.5 mg/g BW) using a phenol red thread (Nguyen et al., ibid.),a modification of the Shirmer test. In brief, the bent end of a small piece of Zone-Quick, Phenol Red Thread (FCI Ophthalmics, Pembrooke, Mass., USA) was placed carefully at the intercanthus of each eye of a resting mouse lightly anaesthetized using inhalation anesthesia isoflurane. The thread was held in place with forceps for 20s, removed from the eye, and the length of the red area measured using the scale provided.

### Histological Assessment of Submandibular Glands

Following euthanasia, whole submandibular salivary glands were surgically removed from each mouse and placed in 10% phosphate-buffered formalin for 24 hrs. Fixed tissues were embedded in paraffin and sectioned at 5-μm thickness. Paraffin-embedded sections were de-paraffinized by immersing in xylene, followed by dehydrating in ethanol. The tissue sections were prepared and stained with hematoxylin and eosin (H&E) dye. Stained sections were observed under a microscope for glandular structure and leukocyte infiltration determination. According to the lymphocytic foci (LF) which were defined as aggregates of >50 leukocytes quantified per each histological section, adjacent sections were used for immunofluorescent staining, as described hereinafter.

### Immunofluorescent Staining for CD3+T Cells and B220+B Cells

Immunofluorescent staining for T and B cells for the infiltrations in the salivary glands was done as previously described (Nguyen et al., ibid.). Briefly histological sections of salivary glands were incubated with rat anti-mouse B220 (BD Pharmingen, San Jose, Calif.) and goat anti-mouse CD3 (Santa Cruz Biotechnology, Santa Cruz, Calif.), followed by incubation with Texas Red-conjugated rabbit anti-rat IgG (Biomeda, Foster City, Calif.) and FITC-conjugated rabbit anti-goat IgG (Sigma-Aldrich, St. Louis, Mo.). The slides were mounted with DAPI-mounting medium (Vector Laboratories, Burlingame, Calif.). Sections were observed at 200× magnification using a Zeiss Axiovert 200M microscope, and images were obtained with AxioVs40 software (Ver. 4.7.1.0, Zeiss) (Carl Zeiss, Thornwood). The number of lymphocytic foci (LF) in each section was blindly enumerated by three individual investigators. Enumeration of B, T cells and total number of nuclei in the LF were performed using Mayachitra imago software (Mayachitra, Inc, Santa Barbara, Calif.).

Immunohistochemical staining for CD11c and F4/80 in salivary glands

Immunohistochemical staining for CD11c or F4/80 was carried out using techniques known to those skilled in the art. In brief, paraffin-embedded salivary glands were deparaffinized by immersion in xylene, followed by antigen retrieval with 10 mM citrate buffer, pH 6.0. Tissue sections were incubated overnight at 4° C. with anti-CD11c or anti-F4/80 antibody (Santa Cruz Biotechnology Santa Cruz, Calif.). Isotype controls were done with rabbit IgG. The slides were incubated with biotinylated goat anti-rabbit IgG followed by horseradish peroxidase-conjugated streptavidin incubation using the Vectastain ABC kit. The staining was developed using diaminobenzidine substrate (Vector Laboratories, Burlingame, Calif.), and counterstaining was performed with hematoxylin. Sections were observed at 200× magnification using a Zeiss Axiovert 200M microscope, and images were obtained with AxioVs40 software (Ver. 4.7.1.0, Zeiss) (Carl Zeiss, Thornwood). Enumeration of CD11c-positive cells or F4/80-positive cells was performed on the entire histological sections of the whole salivary glands using Mayachitra imago software (Mayachitra, Inc, Santa Barbara, Calif.), although lymphocytic infiltrations are normally seen only in the submandibular glands. The results were calculated and expressed as foci per 4 mm (Voulgarelis et al., ibid.). The focus scores were assessed blindly by three different examiners, and the mean scores were determined.

### Determination of Autoantibodies

At the end of the study, sera collected from 30-wk old C57BL/6.NOD-Aec1Aec2 mice were analyzed for autoantibodies against SSA/Ro and SSB/La antibodies. Enzyme-linked immunosorbent assays (ELISA) were developed to detect anti-60-kD multiple antigenic peptide (MAP)-Ro273 antibodies using techniques known to those skilled in the art. A 96-well plate (Nunc, Rochester, N.Y.) was incubated overnight (O/N) with 1 μg MAP-Ro273 (University of Oklahoma Health Sciences Molecular Biology core Facility, Oklahoma City, Okla.) in PBS. The next day, wells were blocked with PBS/0.05% bovine serum albumin (BSA) for 1 hour (hr) at 37° C., fluid was discarded and incubated with 1:100 dilution of serum in blocking buffer for 2 hr at RT. The wells were washed three times with PBS/0.05% Tween and incubated with 1:5000 dilution of goat anti-mouse IgG-HRP (Dako, Carpinteria, Calif.) for 1 hr at room temperature (RT). Thereafter, the wells were washed 3 times, incubated with 1:1 substrate A and B (R&D Systems, Minneapolis, Minn.) for 20 minutes at RT, and the reaction was stopped by stop solution (R&D Systems, Minneapolis, Minn.). The optical density (OD) was measured at 450 nm using a Spectramax M2 plate reader (Molecular Devices Corporation, Sunnyvale, Calif.). The autoantibody against SSB/La (total Ig) was measured by a commercially available ELISA kit (Alpha Diagnostic International, San Antonio, Tex.) according to the manufacturer's protocol.

Detection of Cytokines from Cell Cultures and SG Homogenates

Cytokines from spleen cells and DLN cell culture, serum and homogenates of salivary glands were detected as described previously (Yin et al., 2009, J Neuroimmunol 215, 43-48). Briefly for the cultures, splenocytes and submandibular salivary gland associated draining lymph nodes (DLNs) obtained from treated mice were isolated and cultured in 24-well plates at 5×10⁶ cells/mL RPMI-1640 medium (Invitrogen, Carlsbad, Calif.), containing HL-1 serum replacement (Cambrex Bioscience, Walkersville, Md.), with or without 1 μg/mL Concanavalin A (Con A, Sigma-Aldrich, St. Louis, Mo.). Supernatants were collected after 48 hr incubation. Serum and salivary gland homogenates were prepared as described previously (Vosters et al., ibid.), and original collections were used for detection.

Interleukin-1β (IL-1β), IL-2, tumor necrosis factor-α (TNF-α), IL-12p40 and p70, interferon-γ (IFN-γ), IL-18, IL-17, IL-23, IL-27, IL-6, IL-4, IL-5, IL-13, IL-10, transforming growth factor-β1 (TGF-β1), mast cell proteinase-1 (MCP-1) and macrophage inflammatory proteins-1 (MIP-1) were measured using a multiplex sandwich-ELISA assay (Aushon Biosystem Billerica, Mass.). Duplicates for each sample were tested in three dilutions and the mean values of the duplicates from the optimal dilution were reported (Yin et al., ibid.).

### Statistical Analysis

Differences between two experimental groups were assessed using the unpaired student t-test. Multiple-groups such as multi-cytokine assays were done by one-way ANOVA or Mann-Whitney U test. All analyses were performed with GraphPad Prism statistical software (GraphPad Software Inc. version 4.02, La Jolla, Calif., USA) using a p value ≦0.05 as statistically significant.

### Example 2

### Production of AAV Plasmid Vector Encoding CTLA4IgG

A nucleic acid molecule encoding a CTLA-4 protein comprising the extracellular domain of mouse cytotoxic T-lymphocyte antigen 4 (CTLA4) joined to human Immunoglobulin G (IgG) Cγ1 (CTLA4IgG) was obtained from Dr. Toshimitsu Uede (Institute of Immunological Science, Hokkaido University, Hokkaido, Japan); see Kanaya et al., 2003, Transplantation 75, 275-281, and Nakagawa et al., 1998, Hum Gene Ther 9, 1739-1745, for a description of the CTLA4IgG nucleic acid molecule and production thereof. This nucleic acid molecule was cloned into a recombinant Adeno Associated Virus (AAV) plasmid containing a Cytomegalovirus (CMV) promoter and the Inverted Terminal Repeat (ITRs) sequences for AAV serotype 2 (AAV2). The generated plasmid vector was named pAAV2-CMV-mCTLA4-hIgG (SEQ ID NO:1), also referred to as pAAV2-CTLA4IgG or pAAV-CTLA4IgG.

### Example 3

### Production of AAV Virions Encoding CTLA4IgG

Adenoviral helper packaging plasmid pDG (see, e.g., Smith et al., 2002, Biotechniques 33, 204-206, 208, 210-211; Grimm et al., 2003, Mol Ther 7, 839-850) was used to generate AAV serotype 2 virions encoding CTLA4Ig protein CTLA4IgG (AAV2-CTLA4IgG virions). Plates (15 cm) of ˜40% confluent 293 T cells were cotransfected with either pAAV-LacZ or pAAV-CTLA4IgG according to standardized methods (Kanaya et al., ibid.). Clarified cell lysates were adjusted to a refractive index of 1.372 by addition of CsCl and centrifuged at 38,000 rpm for 65 hr at 20° C. Equilibrium density gradients were fractionated and fractions with a refractive index of 1.369-1.375 were collected. The titer of DNA physical particles in AAV-CTLA4IgG virion stocks was determined by Q-PCR (see, for example, Schmidt et al., 2004, J Virol 78, 6509-6516), and the virions were stored at −80° C. On the day of AAV-CTLA4IgG virion administration to C57BL16.NOD-Aec1Aec2 mice, the virion was dialyzed for 3 hr against saline.

### Example 4

### CTLA4IgG Inhibited B7 Expression of Macrophages In Vitro

Expression and biological activity of the CTLA4IgG fusion protein were confirmed by western blot and blocking the B7:CD28 pathway in vitro, respectively, prior to assessing stable expression of CTLA4IgG in the salivary glands of C57BL/6.NOD-Aec1Aec2 mice.

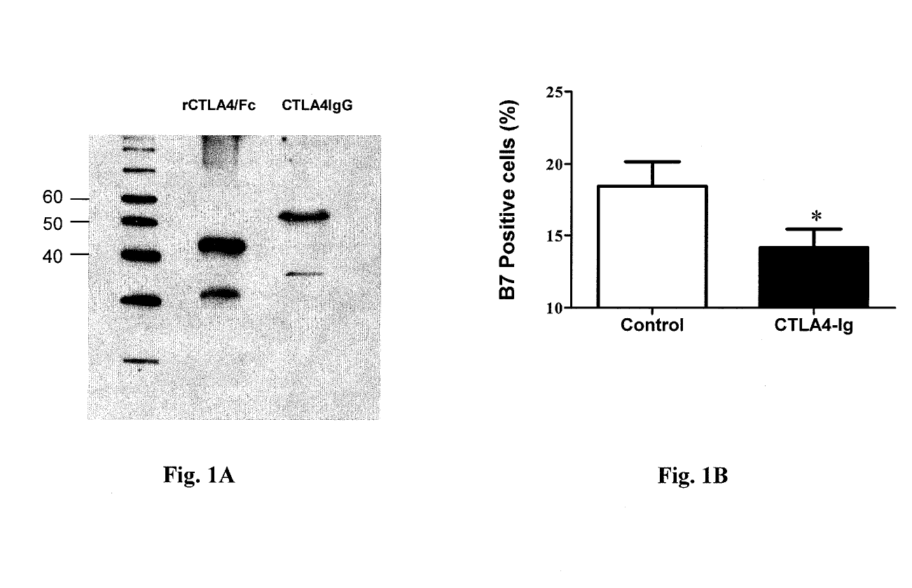

Fusion of the Cγ1 domain of IgG to the sCTLA4 domain resulted in a chimeric protein of approximately 62 kDa. FIG. 1A shows that the recombinant protein could easily be detected in the media of transfected 293 cells.

Macrophages, similar to dendritic cells, are one of the professional antigen presenting cells (APCs) that express costimulatory molecule B7, which can bind to CD28 on T cells during antigen presentation. To test for the ability of the recombinant CTLA4IgG to bind and block B7 detection, supernatant from CTLA4IgG-expressing cells was pre-incubated with macrophages, and then B7 expression was quantified by flow cytometry assay. In the absence of CTLA4IgG, about 18% of macrophages cells expressed B7. CTLA4IgG expression significantly down-regulated B7 expression to about 14% (P=0.04), as demonstrated in FIG. 1B. These results demonstrate that the expressed CTLA4IgG was able bind B7 and down-regulate B7 activity in vitro.

### Example 5

### CTLA4IgG Expressed in C57BL/6.NOD-Aec1Aec2 Mice In Vivo

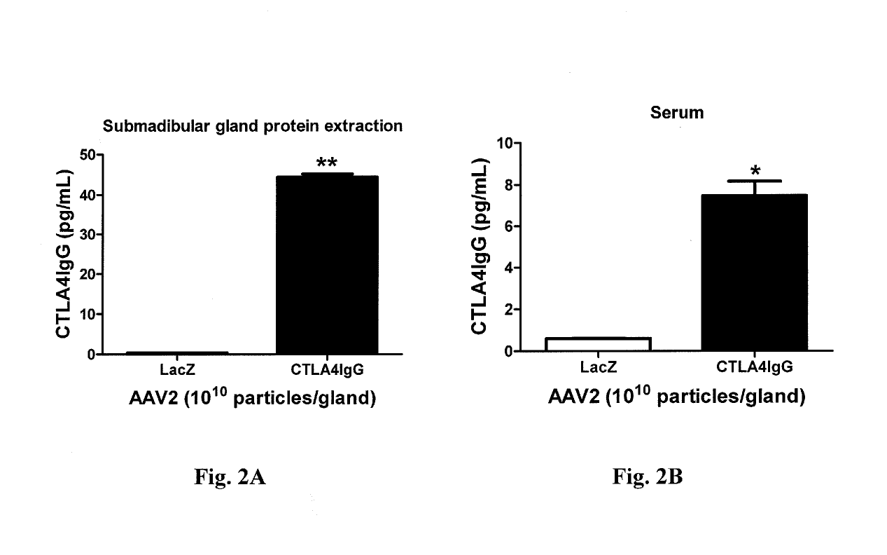

To confirm the stable expression of CTLA4IgG in vivo after local delivery of AAV virion AAV2-CTLA4IgG to the salivary glands of C57BL16.NOD-Aec1Aec2 mice, homogenates of salivary glands and sera were obtained at the end of the study, when the mice were 30 weeks old; the homogenates were pooled according to each group. Using a sandwich-ELISA to detect the recombinant chimeric protein (mouse CTLA4 and human IgG), mice that received AAV virions encoding CTLA4IgG had much higher levels of CTLA4IgG (44.5±0.76 pg/mL in salivary glands, and 7.48±0.70 pg/mL in serum) (mean±SD) compared with mice that received a virion expressing LacZ (0.39±0.02 pg/mL in salivary glands and 0.62±0.01 pg/mL in serum; P=0.0003 and P=0.0102 respectively in salivary gland and serum), demonstrating the recombinant protein CTLA4IgG was expressed in vivo; see FIG. 2.

### Example 6

### Local Delivery of AAV2-CTLA4IgG Protects Function of Salivary Glands in C57BL/6.NOD-Aec1Aec2 Mice

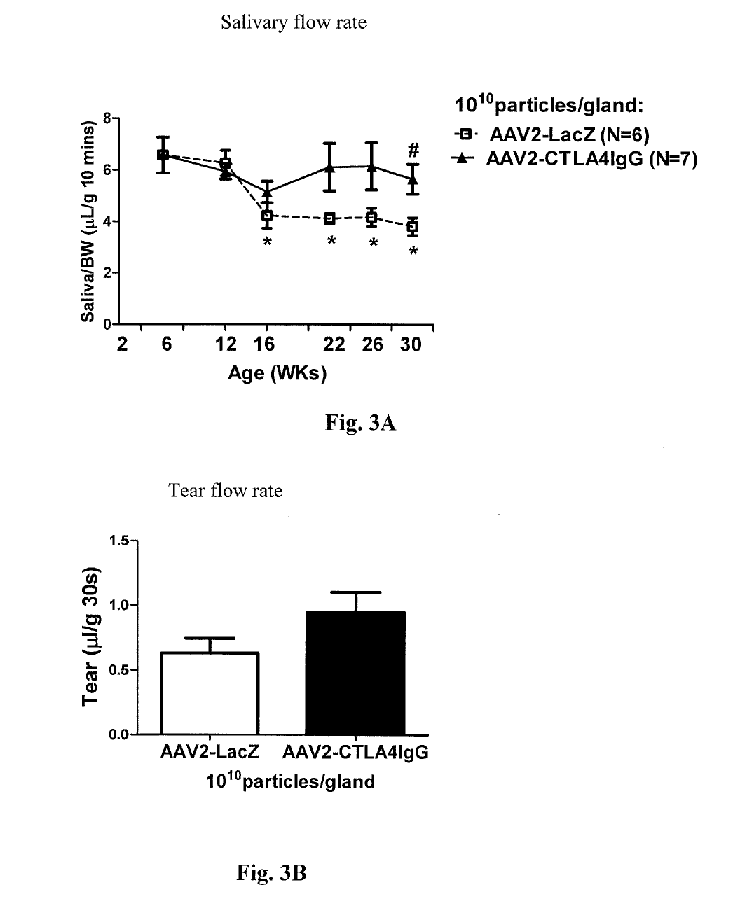

To better understand the effect of CTLA4IgG on salivary gland function, stimulated saliva flow was measured in both treated and control mice over time. In agreement with previous studies (Cha et al., ibid.), mice treated with the control AAV2-LacZ virion had a significant decrease of saliva flow (4.25±0.64 μL/g 10 mins), compared to the baseline (6 weeks, 6.10±0.30 μL/g 10 mins) by 16 weeks that continued to decline over time (Nguyen et al., ibid.). The mice transduced with AAV virion AAV2-CTLA4IgG also showed a drop of the saliva flow at 16 weeks (5.13±1.22 μL/g 10 mins), but it was not statistically significant compared with the 6-week baseline. Furthermore, by 22 weeks the saliva flow from mice administered AAV2-CTLA4IgG had recovered to near baseline values (6.13±0.92 μL/g 10 mins) which was sustained for the remainder of the study and was statistically different compared with the AAV2-LacZ treated group at 30 weeks (P=0.02). These data, depicted in FIG. 3A, demonstrate that expression of CTLA4IgG in the salivary glands of C57BL/6.NOD-Aec1Aec2 mice can prevent loss of salivary gland function.

### Example 7

### Salivary Gland Transduction with AAV2-CTLA4IgG Showed an Affect on Lachrymal Gland Dysfunction in C57BL/6.NOD-Aec1Aec2 Mice

Previous research has suggested that following cannulation of the salivary gland, greater than 90% of the AAV2 vector remains in the gland, with some vector detected in the liver and spleen (Katano et al., 2006, Gene Therapy 13, 594-601). In order to test if local delivery of AAV2-CTLA4IgG had distal effects on the loss of lachrymal gland function in the C57BL/6.NOD-Aec1Aec2 mice, tear flow was measured at the end of the study when the mice were 30 weeks old. CTLA4IgG expression in the salivary gland showed an overall increase in tear flow compared with the lacZ-expressing group in FIG. 3B (P=0.1316).

### Example 8

### Salivary Gland Transduction with AAV2 CTLA4IgG can Affect Immune Infiltrates

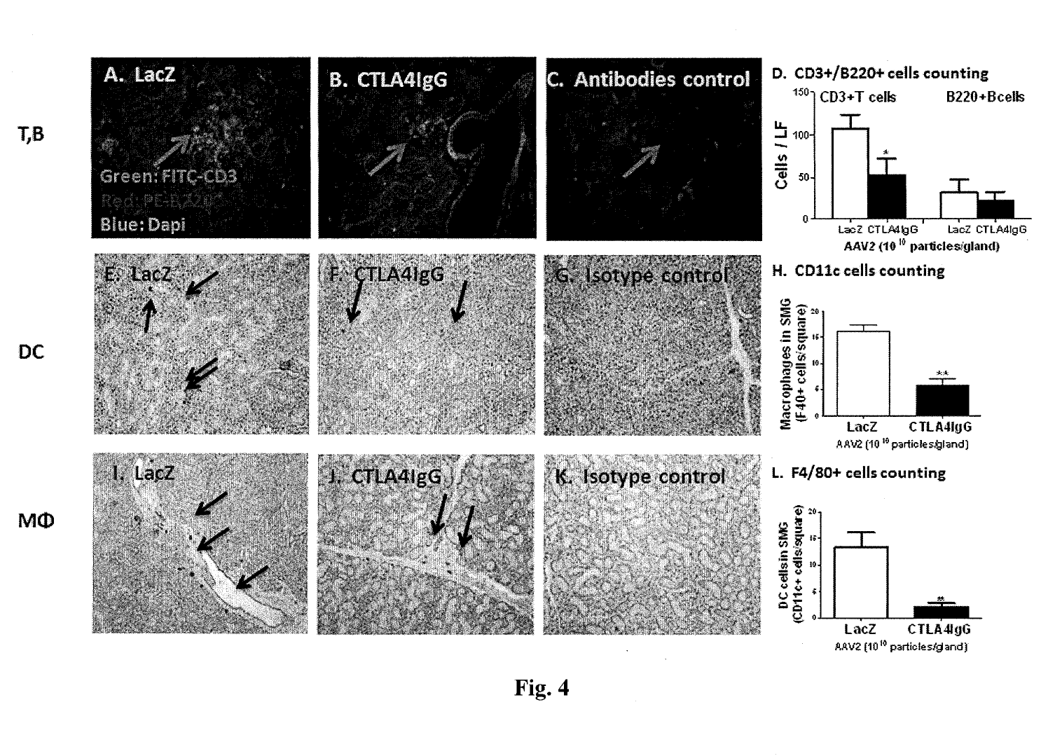

To determine the effect of CTLA4IgG on the lymphocyte foci in the salivary glands, the number of LFs as well as the number of T and B cells within the gland were detected by immunofluorescent staining of CD3 and B220 respectively, as shown in FIG. 4A, FIG. 4B, FIG. 4C, and FIG. 4D. The number of LF was decreased in salivary glands from mice administered AAV virionAAV2-CTLA4IgG (0.71LF/per gland) compared with control mice administered AAV2-LacZ (2.16 LF/per gland). Furthermore, the number of T and B cells present in the LF of the AAV2-CTLA4IgG treated mice also decreased; although the decrease in T cells was significant, the change in B cells was not statistically significant compared with control AAV2-LacZ treated mice (P=0.0464 and P=0.3024 for analysis of T and B cells respectively) (FIG. 4C, FIG. 4D, FIG. 4E, and FIG. 4F).

Macrophages are crucial in activation of T cells and function as non-professional antigen presenting cells as well as auto antigen presentation in autoimmune disease (Kulkarni et al., 1991, Immunology and Cell Biology 69, 71-80). Thus, DC and macrophage number was analyzed by staining CD11c+ DCs and F4/80+ cells, respectively, in the salivary glands from C57BL/6.NOD-Aec1Aec2 mice. Staining for CD11c and F4/80 indicated a significant decrease in the number of DCs and macrophages in CTLA4IgG expressing mice compared with the LacZ control group, as shown in FIG. 4E, FIG. 4F, FIG. 4G, FIG. 4H, FIG. 4I, FIG. 4J, FIG. 4K, and FIG. 4L; (DCs: 13.33±2.78/gland in LacZ group vs 2.14±0.70/per gland in CTLA4IgG mice, P≦0.01. macrophages: 14.89±0.11/gland in LacZ group vs 3.84±2.31/per gland in CTLA4IgG mice, P≦0.0001). These data indicate that local expression of CTLA4IgG can inhibit macrophage migration and inhibit accumulation of T-cells, DCs, and macrophages in the salivary glands.

### Example 9

### CTLA4IgG Expression does not Change Autoantibody Levels in C57BL/6.NOD-Aec1Aec2 Mice

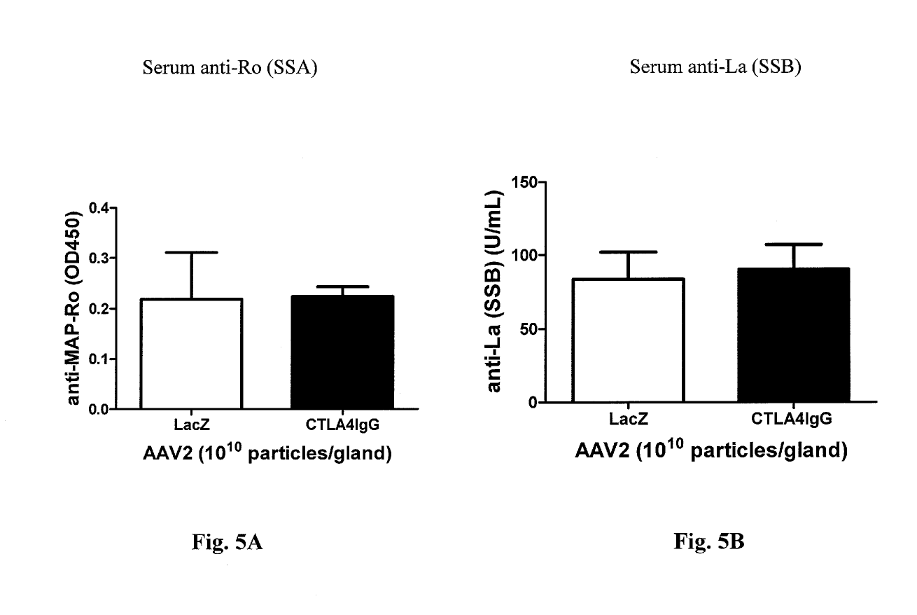

To observe the effect of CTLA4IgG on the regulation of systemic B cell activation, the amount of anti-Ro (SSA) and anti-La (SSB), autoantibodies was measured, the presence of such autoantibodies being highly correlated with pSS (Whittingham et al., 1987, Lancet 2, 1-3). FIG. 5 indicates that little change in anti-Ro or anti-La antibody titer was detected in the AAV2-CTLA4IgG treated group compared with the AAV2-LacZ treated mice.

### Example 10

### Deactivation of T Cells and Macrophages, and Activation of Treg by CTLA4IgG are Found in Salivary Gland Associated Lymph Nodes

Previous research has suggested that proinflammatory T cell cytokines can trigger cell death in salivary gland cells and block calcium signaling (Wu et al., 1996, American Journal of Physiology 270, C514-521). Furthermore, several reports have noted changes in Th1 and Th17 cytokines in Sjögren's patients or animal model (Sakaguchi et al., ibid.; Leung et al., 2010, Cellular and Molecular Immunology 7, 182-189). Meanwhile a down-regulation of suppressive Treg was noted in the salivary gland of pSS patients (Li et al., 2007, Journal of Rheumatology 34, 2438-2445). To investigate the effect of CTLA4IgG expression on local and systemic T cell responses, cytokine levels were measured in different populations of T cells or macrophages in both systemic organs of spleen and serum, as well as locally in the SG and associated draining lymph nodes.

Serum, splenocytes, and salivary gland DLNs were collected at the end of the study as described in the Examples herein. Splenocytes and DLN cells were pooled according to vector treatment group. Culture supernatants were collected following incubation with or without ConA for 48 hrs as indicated in the Examples herein. Cultures and serum were then analyzed for levels of the indicated cytokines (in pg/mL) by multi-cytokine assay in triplicate. For cell cultures, values are the mean of ConA treated cells (triplicate) subtracted from media alone background. For serum, values are the mean of each group of mice (AAV2-LacZ (n=6) and with AAV virion AAV2-CTLA4IgG (n=7) respectively). Results are provided in Table 1.

TABLE 1
Local and systemic T cell cytokine production in AAV virion-treated mice (pg/mL).

| | | DLN cells | | Spleen cells | | Serum | |
|---|---|---|---|---|---|---|---|
| | | LacZ | CTLA4IgG | LacZ | CTLA4IgG | LacZ | CTLA4IgG |
| Th1- | IL-12p70 | 12 | 0.00↓ | 4.6 | 0.20 | 88.5 ± 15.8 | 88.5 ± 15.8 |
| | IFN-γ | 3.8 | 0.00↓ | 122 | 115.0 | 872.6 ± 1276.8 | 457.2 ± 371.8↓ |
| | IL-18 | 11 | 0.00↓ | 16 | 0.00↓ | 694 ± 562 | 394.10↓ |
| Th17- | IL-17 | N/A | N/A | 2.1 | 0.00↓ | 2.8 ± 3.3 | N/A↓ |
| | IL-23 | 14 | N/A↓ | 78.2 | 36.10↓ | 335.6 ± 365.7 | 101.99↓ |
| Treg- or SG | TGF-β1 | N/A | 41410↑ | 2084 | 1384 | 1501176 ± 277249 | 1260620 ± 289478 |
| epithelial cells | | | | | | | |
| Non-specific | IL-6 | 2452 | 1688↓ | 2557 | 2407 | 309 ± 1241 | 289 ± 355 |
| (Macrophages) | | | | | | | |
| | TNF-α | 43 | 27↓ | 98 | 120 | 37 ± 83 | 41 ± 142 |
| Chemokines | MCP-1 | 10 | 2↓ | 44 | 43 | 63 ± 19 | 66 ± 26 |
| | MIP-1α | 224 | 130↓ | 357 | 237 | 2 ± 0.2 | 2.5 ± 2.5 |

Unpaired student t-test was used for statistical analysis.
↑Production of cytokine from AAV2-CTLA4IgG group is ≧50% higher than from AAV2-LacZ group.
↓Production of cytokine from AAV2-CTLA4IgG group is ≧50% less than from AAV2-LacZ group.
N/A: not detectable by assay method used

In the salivary gland homogenates, only down-regulation of IL-6 was observed in the AAV2-CTLA4IgG treated mice (mean=169.60 pg/mL) compared with AAV2-LacZ controls (mean=86.51 pg/mL), which was not statistically significant (P=0.9062) (data not shown). Interestingly a more than 50% increase of TGF-β1 production (mean=1208.70 pg/mL in CTLA4IgG group compared to mean=804.53 pg/mL in LacZ group) was detected; however the difference was not statistically significant (P=0.093). It is known that TGF-β1 is a crucial cytokine released from activated Treg (Fehervari et al., 2004, Journal of Clinical Investigation 114, 1209-1217). These data demonstrate an activation of Treg by CTLA4IgG local expression locally in the salivary glands. A general down-regulation of ConA stimulated cytokine productions in AAV2-CTLA4IgG treated mice compared with control AAV2-LacZ mice was observed in the culture media for the DLN cells associated with the salivary gland. Th1 cytokines (IL-12, IFN-γ and IL-18) and Th17 cytokines (IL-23 and IL-6) were all down-regulated. TGF-β1 was strikingly up-regulated. In addition, non-specific proinflammatory cytokines, IL-6 and TNF-α, as well as chemokines MCP-1 and MIP-1α, which are mainly released from macrophages, were decreased. Little change was detected in Th2 cytokines such as IL-4, IL-5, and IL-13 after local expression of CTLA4IgG (data not shown). These data demonstrate that in the SG associated DLNs, CTLA4IgG expression can deactivate proinflammatory Th1 and Th17 cells but stimulate suppressive nTreg cells, showing that CTLA4IgG can shift T cell response from proinflammatory Th1/Th17 to suppressive nTreg. These data demonstrate that CTLA4IgG expression can reduce proinflammatory cytokines released by Th1, Th17 cells, DCs, and macrophages, while stimulating production of anti-inflammatory cytokines such as TGF-beta1. Together with the data showing down-regulation of DCs and macrophages by CTLA4IgG (FIG. 4), these data further support a decrease in macrophages following CTLA4IgG treatment.

### Example 11

### Effect of CTLA4IgG on Regulation of Systemic T Cell Response in the C57BL/6.NOD-Aec1Aec2 Mice

Distal affects following local gene therapy have been reported and are hypothesized to be the result of circulating levels of recombinant protein (Ghivizzani et al., 1998, Proc Natl Acad Sci 95, 4613-4618). To test for changes in the systemic immune system, cytokines were also measured in the serum and in spleen cell cultures. Results are reported in Table 1. Wide variation was seen in cytokine values in serum, and none of the cytokine levels from the CTLA4IgG and LacZ groups showed statistical significance. However, a decrease in a majority of cytokines associated with Th1 and Th17 cells, such as IFN-γ, IL-18, IL-17 and IL-23, were seen in the serum from AAV2-CTLA4IgG treated mice compared with AAV2-LacZ treated mice. Meanwhile production of IL-12, IL-18, IL-17 and IL-23 from splenocyte cultures challenged by ConA, was down-regulated in the AAV2-CTLA4IgG treated group compared with the AAV2-LacZ treated mice. Unlike the local immune response, little change in nonspecific cytokines, chemokines, or TGF-β1 was detected in serum or cultured splenocytes. These data imply that CTLA4IgG expression can also decrease proinflammatory cytokines in the peripheral immune system following local salivary gland gene transfer in C57BL16.NOD-Aec1Aec2 mice.

### Example 12

### Conclusions and Discussion

These Examples indicate results from the study of the immunomodulatory potential of CTLA-4 to treat the SS-like phenotype in the C57BL/6.NOD-Aec1Aec2 mouse model. Expression of a recombinant CTLA-4 and immunoglobulin-G (IgG) fusion protein (CTLA4IgG) locally in the salivary glands of mice using AAV vectors resulted in the prevention of the age dependent loss of salivary gland function observed in control mice treated with an AAV vector expressing beta galactosidase. Lachrymal gland function was also improved compared with control mice (P=0.1316). Intra-glandular staining found a decreased number and size of lymphocyte foci (LF), along with trend of decrease of T, B infiltrations and macrophages in the salivary glands. Further immunological studies also indicated a decrease in T cell and macrophages proinflammatory cytokines and an increase in the Treg produced cytokine TGF-b1 both locally and systemically.

In autoreactive T cell-initiated autoimmune diseases such as rheumatoid arthritis, the blocking of antigen presentation and deactivation of proinflammatory T lymphocytes, is central in the treatment of these diseases (Lehmann et al., 1992, Nature 358, 155-157). Recombinant proteins such as CTLA4-immunoglublin (ORENCIA, Abatacept) have demonstrated clinical utility in treating this condition by shutting down T cell activation by blocking the B7:CD28 costimulatory pathway thus inhibiting the auto antigen presentation (Perkins et al., ibid.), but enhancing nTreg function (Wing et al., ibid.; Takahashi et al., ibid). In this study, expression of CTLA4IgG by gene transfer of AAV2 virions locally in the salivary glands of C57BL/6.NOD-Aec1Aec2 mice resulted in long term protection of salivary gland function.

In Sjögren's syndrome, activated CD4+ T lymphocytes including Th1 and Th17 cells infiltrate the salivary and lachrymal glands and produce a variety of proinflammatory cytokines, such as IFN-γ and IL-17, which may trigger gland damage (Tsunawaki et al., 2002, J Rheumatol 29, 1884-1896). This event may represent a crucial stage in the pathogenesis in SS ((Voulgarelis et al., ibid.) Changes in the systemic and local immune system, spleen, serum and DLNs, can accompany the immune activation in the exocrine glands.

The data provided herein indicated that, in the C57BL/6.NOD-Aec1Aec2 mouse, CTLA4IgG expression in the gland triggers a pattern of down-regulation of both T and B lymphocytes infiltration in the salivary glands. This effect is accompanied with a consistent overall decrease of Th1- and Th17-cytokines. Specifically, the tests in the spleen and DLN cells were triggered by ConA, a strong non-specific antigen to stimulate T cell activation (Palacios, 1982, J Immunol 128, 337-342). This strongly demonstrates that expression of CTLA4IgG locally in the salivary glands can deactivate the proinflammatory T cells, especially during the activation process, in both systemic as well as local correlated immune systems. More importantly, a significant increase in TGF-β1 expression in both the salivary glands and the DLNs indicates an activation of nTreg, which implies an altered T cell response from proinflammatory to suppressive T cells as the mechanism associated with the protective effect of CTLA4IgG expression in this study.

Moreover, a trend of decrease of B cells in the LF and significant down-regulation of macrophages in the salivary glands were found in the CTLA4IgG obtained mice, accompanied with evidence of deactivation of macrophages in the DLNs. Both macrophages as well as B lymphocytes are non-professional antigen presenting cells that are required in antigen presenting and subsequently T cell activation (Kulkarni et al., ibid.). It is also noted that recombinant CTLA4IgG has an extended deactivation to macrophages (Cutolo et al., ibid) and B lymphocytes (Izawa et al., ibid.), which is in agreement with what was observed in this study.

In conclusion, the data support prevention of primary Sjögren's syndrome from AAV2 mediated CTLA4IgG by local gene delivery in the salivary glands from the tested animal model, which strongly demonstrates a potential treatment target for this disease. The underlining pathway for this effect is through tilting the T cell autoimmunity to the suppressive T cells and archive immune tolerance.

### Example 13

### Additional Conclusions and Discussion

In this study, local expression of CTLA4IgG by gene transfer to the salivary glands of C57BL/6.NOD-Aec1Aec2 mice, a pSS animal model, resulted in a decrease in the sialadenitis and improvement in gland function compared with mice that received a control vector.

The advantage of localized gene transfer is to direct the expression of the therapeutic molecule to the site of maximum effect while minimizing the systemic complications that can be associated with off target effects. Using this approach the inventors were able to achieve much higher local concentrations of CTLA4IgG in the salivary glands compared to circulating levels in the serum. The data further confirm that ductal cells within the gland represent a good depot site for production of recombinant proteins (Cotrim et al., 2008, Toxicol Pathol 36, 97-103. Indeed, previous experiments have demonstrated expression from salivary gland ductal cells for the life of the animal (Voutetakis et al., 2004, Proc Natl Acad Sci 101, 3053-3058).

In both patients and C57BL/6.NOD-Aec1Aec2 mice, activated CD4+ T lymphocytes including Th1 and Th17 cells infiltrate the salivary and lachrymal glands, and produce a variety of proinflammatory cytokines, such as IFN-gamma and IL-17, which may trigger gland damage and represent a crucial element in the pathogenesis of pSS (Voulgarelis et al, ibid.; Tsunawaki et al., ibid). The inventors detected a decrease in Th17 cytokine in both the DLN and spleen following expression of CTLA4IgG, suggesting a corrective shift in this critical cell population.

Besides the negative effect on T cells as a result of blockade of the B7:CD28 costimulatory pathway (Moreland et al., ibid.), it is also noted that recombinant CTLA4IgG may directly or indirectly deactivate DCs, macrophages and B lymphocytes (Takahashi et al., ibid.; Cutolo et al., ibid). The data indicate that in C57BL/6.NOD-Aec1Aec2 mice, CTLA4IgG expression results in a decrease in T and B lymphocytes as well as DCs and macrophages in the salivary glands that is accompanied by a down-regulation in proinflammatory cytokines. This finding is in agreement with previous reports on the effect of CTLA4IgG in other autoimmune disease models (Izawa et al., ibid., Cutolo et al., ibid.).

Interestingly, a significant increase in TGF-beta1 expression in both the salivary glands and the DLNs was observed. The increase in TGF-beta1 expression may be related to an increase in nTreg or negative regulation of epithelial cells by CTLA4IgG (Takahashi et al., ibid.). In addition to its role in the immune system, TGF-beta1 expression was found to be important in maintaining epithelial tight junctions, an important component in the fluid movement of salivary glands (Howe, et al., 2005, Am J Pathol 167, 1587-1597) and therefore may be directly related to the improvement in saliva flow.

These studies demonstrate an improvement of salivary gland function, which could result from the inhibition of sialadenitis after local expression of CTLA4IgG. This result identifies local delivery of AAV2-CTLA4IgG as a treatment of pSS. In addition, some improvement in lachrymal gland was also observed. This difference is likely related to the lower circulating levels of CTLA4IgG in the serum compared with the levels in the salivary gland. Despite the positive results achieved in this study, the circulating levels of CTLA4IgG are well below those clinically used with abatacept. Further increases in the dose of AAV virions or the use of AAV virions with improved gene transfer activity in the salivary gland are likely to result in higher circulating levels and may have a more significant impact on extraglandular manifestations of Sjögren's syndrome.

In summary, the data provided herein demonstrate that inhibition of the costimulatory pathway CD28 by expression of CTLA4IgG locally in the salivary gland can be a useful approach for reducing inflammation and improving the secretory activity associated with Sjögren's syndrome.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims.

1. A method to protect a subject from Sjögren's syndrome, the method comprising administering to the subject an adeno-associated virus (AAV) virion comprising an AAV vector that encodes a soluble CTLA-4 (sCTLA-4) protein, wherein administration of the virion protects the subject from Sjögren's syndrome.

2. The method of claim 1, wherein the virion is administered to a salivary gland or a lachrymal gland of the subject.

3. (canceled)

4. The method of claim 1, wherein the sCTLA-4 protein comprises a sCTLA-4 fusion protein, wherein the sCTLA-4 protein domain is joined to a fusion segment.

5. The method of claim 4, wherein the fusion segment is an immunoglobulin fusion segment.

6. The method of claim 4, wherein the fusion segment is an IgG Cγ1 immunoglobulin fusion segment.

7. (canceled)

8. The method of claim 1, wherein the sCLTA-4 protein comprises an amino acid sequence at least 80% identical to SEQ ID NO:4.

9. The method of claim 1, wherein the AAV vector has nucleic acid sequence SEQ ID NO:1.

10. (canceled)

11. The method of claim 1, wherein administration of the virion maintains salivary or lachrymal gland function at a level equivalent to the level of salivary or lachrymal gland function prior to administration of the virion.

12. (canceled)

13. The method of claim 1, wherein administration of the virion improves the level of salivary gland or lachrymal gland function.

14. (canceled)

15. The method of claim 1, wherein administration of the virion reduces immune cell infiltration in the salivary or lachrymal glands of a subject with Sjögren's syndrome.

16-17. (canceled)

18. An AAV vector that encodes a fusion protein comprising a sCTLA-4 protein and an immunoglobulin fusion segment.

19. (canceled)

20. The AAV vector of claim 18, wherein the vector has nucleic acid sequence SEQ ID NO:1.

21. An AAV virion that comprises the AAV vector of claim 18.

22. (canceled)

23. The AAV vector of claim 18, wherein the fusion segment is an IgG Cγ1 immunoglobulin fusion segment.

24. (canceled)

25. A nucleic acid molecule comprising an AAV vector of claim 18.

26. A composition for treating or preventing Sjögren's syndrome comprising an AAV virion comprising an AAV vector that encodes a sCTLA-4 protein, wherein administration of the treatment to a subject protects the subject from Sjögren's syndrome.

27. (canceled)

28. The composition of claim 26, wherein the sCTLA-4 protein comprises a sCTLA-4 fusion protein, wherein the sCTLA-4 protein domain is joined to a fusion segment.

29. The composition of claim 28, wherein the fusion segment is an immunoglobulin fusion segment.

30. The composition of claim 28, wherein the fusion segment is an IgG Cγ1 immunoglobulin fusion segment.

31-35. (canceled)

36. The composition of claim 28, wherein the sCLTA-4 protein comprises an amino acid sequence at least 80% identical to SEQ ID NO:4.


SEQUENCE LISTING (5 sequences)

SEQ ID NO: 1 - DNA, 5922 bp, Artificial Sequence
  Synthetic sequence
catggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatccggtcgcgcgaattctgcagatatccatcacactggcggccgctcgagcaccaatgaagttcctctctgcaagagacttccatccagttgccttcttgggactgatgctggtgacaaccacggccttcgccatacaggtgacccaaccttcagtggtgttggctagcagccatggtgtcgccagctttccatgtgaatattcaccgtcacacaacactgatgaggtccgggtgactgtgctgcggcagacaaatgaccaagtgactgaggtctgtgccacgacattcacagagaagaatacagtgggcttcctagattaccccttctgcagtggtacctttaatgaaagcagagtgaacctcaccatccaaggactgagagctgttgacacgggactgtacctctgcaaggtggaactcatgtacccaccgccatactttgtgggcatgggcaacgggacgcagatttatgtcattgatccagaaccatgcccggattctgaggatcccgagggtgagtactaagcttcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgsgacggccgcgactctagagtcgacctgcaggcatgcaagcttgggatctttgtgaaggaaccttacttctgtggtgtgacataattggacaaactacctacagagatttaaagctctaaggtaaatataaaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcgcggcctaatggttacaaataaagcaatagcatcacaaatttcacaaataaagcatttttttcactgcattctagttgtggtttgtccaaactcatcaatgtatcttatcatgtctggatcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaagatctctagaggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaagaattcgcgcgtccggatcgatccgggcaacgttgttgccattgctgcaggcggagaactggtaggtatggaagatctttggccactccctctctgcgcgctcgctcgctcactgaggccgggcgaccaaaggtcgcccgacgcccgggcttggcggcctcagtgagcgagcgagcgcgcagagagggagtggccaactccatcactaggggttcctggaggggtggagtcgtgacgtgaattacgtcatagggttagggaggtcttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattac

SEQ ID NO: 2 - protein, 149 aa, Artificial Sequence
  Synthetic Construct
MKFLSARDFHPVAFLGLMLVTTTAFAIQVTQPSVVLASSHGVASFPCEYSPSHNTDEVRVTVLRQTNDQVTEVCATTFTEKNTVGFLDYPFCSGTFNESRVNLTIQGLRAVDTGLYLCKVELMYPPPYFVGMGNGTQIYVIDPEPCPDS

SEQ ID NO: 3 - DNA, 369 bp, Artificial Sequence
  Synthetic sequence
atacaggtgacccaaccttcagtggtgttggctagcagccatggtgtcgccagctttccatgtgaatattcaccgtcacacaacactgatgaggtccgggtgactgtgctgcggcagacaaatgaccaagtgactgaggtctgtgccacgacattcacagagaagaatacagtgggcttcctagattaccccttctgcagtggtacctttaatgaaagcagagtgaacctcaccatccaaggactgagagctgttgacacgggactgtacctctgcaaggtggaactcatgtacccaccgccatactttgtgggcatgggcaacgggacgcagatttatgtcattgatccagaaccatgcccggattct

SEQ ID NO: 4 - protein, 123 aa, Artificial Sequence
  Synthetic Construct
IQVTQPSVVLASSHGVASFPCEYSPSHNTDEVRVTVLRQTNDQVTEVCATTFTEKNTVGFLDYPFCSGTFNESRVNLTIQGLRAVDTGLYLCKVELMYPPPYFVGMGNGTQIYVIDPEPCPDS

SEQ ID NO: 5 - protein, 124 aa, Homo sapiens [sequence omitted]